(19) United States
(12) Patent Application Publication
(10) Pub. No.: US 2023/0193199 A1
(43) Pub. Date: Jun. 22, 2023

(54) Methods for Printing Functional Human Neural Tissue

(71) Applicant: Wisconsin Alumni Research Foundation, Madison, WI (US)

(72) Inventors: Su-Chun Zhang, Madison, WI (US); Yuanwei Yan, Madison, WI (US)

(21) Appl. No.: 18/057,026

(22) Filed: Nov. 18, 2022

Related U.S. Application Data
(60) Provisional application No. 63/280,886, filed on Nov. 18, 2021.

(51) Int. Cl. C12N 5/0793

(52) U.S. Cl. CPC C12N 5/0619; C12N 2506/08; C12N 2501/13; C12N 2501/70; C12N 2501/42

(57) ABSTRACT

The disclosure generally relates to methods and compositions for preparing a neural tissue construct. In particular, provided herein are methods for generating a neural tissue construct using glutamatergic cortical progenitor cells; GABAergic interneuron progenitor cells; and bio-ink.

### CROSS-REFERENCE TO RELATED APPLICATIONS

This application claims priority to U.S. provisional application No. 63/280,886, filed Nov. 18, 2021, the disclosure of which is expressly incorporated by reference herein.

### REFERENCE TO A SEQUENCE LISTING

A computer readable form is filed with this application by electronic submission and is incorporated into this application by reference in its entirety. The computer readable form is contained in the first file created on Nov. 17, 2022, having the file name “Muscle_Contraction1.avi” and is 122 megabytes in size, and the second file created on Nov. 17, 2022, having the file name “Muscle Contraction2.avi” and is 88 megabytes in size.

### FIELD OF THE DISCLOSURE

The disclosure generally relates to methods and compositions for preparing a neural tissue construct, particularly methods to fabricate brain-regional specific tissues with functional connectivity.

### BACKGROUND

The human brain forms by specialized neuronal and glial cells, which form a neural network for fundamental functions including memory, consciousness, language and thought. Studying neuronal-glial networks is essential to probing neurological diseases including Alzheimer's (AD) Parkinson's (PD), amyotrophic lateral sclerosis (ALS), and autism spectrum disorders (ASD).

Conventionally, such studies have proven to be challenging due to the lack of a reliable model of living human neural tissues. Brain organoids, generated from human pluripotent stem cells (hPSCs), including induced pluripotent stem cells (iPSCs) or embryonic stem cells (hESCs), present a potential solution. However, the formation of functional neural networks in organoids often takes months or even over a year, and the circuits formed between the assembloids are so far randomly positioned. A potential way to complement the organoid system is 3D bioprinting, which enables the assembly of human tissues by spatially controlling the deposition of living cells and hydrogels to generate biologically complex cytoarchitecture. The printing of neural tissues has been explored but the printed tissues do not show functional neuronal networks.

Thus, there remains a need in the art for reagents and methods to produce a platform for assembling neural tissues rapidly and reliably that comprise neuronal and glial subtypes and form functional networks between neurons and between neurons and glia therein.

### SUMMARY OF THE DISCLOSURE

This disclosure sets forth reagents and method for assembling neural tissues comprising neuronal and glial subtypes that form functional networks between neurons and between neurons and glia rapidly, i.e., within weeks. This disclosure provides methods and compositions for preparing a neural tissue construct, particularly for fabricating brain-regional specific tissues with functional connectivity. In particular, provided herein are methods for generating a neural tissue construct using glutamatergic cortical progenitor cells; GABAergic interneuron progenitor cells; and bio-ink. The invention further provides therapeutic methods to model human neural network for Alexander disease (AxD) and other neurodegenerative disorders and screening for therapeutics for AxD and other neurodegenerative disorders.

Provided herein is a method of generating a neural tissue construct, comprising:

(a) horizontally depositing by bioprinting on a suitable surface a band comprising a mixture, wherein the mixture comprises

(i) glutamatergic cortical progenitor cells;

(ii) GABAergic interneuron progenitor cells; and

(iii) bio-ink

(b) repeating the depositing step of (a) a plurality of times to form a plurality of bands; and
(c) maturing the deposited plurality of bands to allow the cells to form a neural tissue construct.

Also provided herein is a method of method of screening a test agent, the method comprising: (a) depositing a test agent on the neural tissue construct disclosed herein; (b) measuring a functional parameter of the contacted neural tissue construct; and (c) comparing the functional parameter to that parameter measured in neural tissue construct which has not been contacted with the test agent, wherein modulation of the functional parameter after contact with the test agent indicates the test agent is a candidate therapeutic agent.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1A-FIG. 1B show the immunofluorescence and quantification of neuronal markers FOXG1 and PAX6 for the differentiated cortical progenitors from hPSCs at day 21.

FIG. 2A-FIG. 2B show the immunofluorescence and quantification of markers NKX2.1 and GABA for the differentiated GABA interneuron progenitors from hPSCs at day 21.

FIG. 3A-FIG. 3B show the immunofluorescence and quantification of markers S1000 and GFAP for the differentiated astrocyte progenitors from hPSCs.

FIG. 4 shows the gelation time of different composition of fibrinogen and thrombin. At a constant concentration of fibrinogen (5 or 2.5 mg/mL), the gelation time decreased with the increased level of thrombin. At 0.5 U thrombin, the gelation time also reduced with increasing fibrinogen concentrations.

FIG. 5 shows a schematic outlining the design of culture neural progenitor cells (NPCs) in the fibrin gel.

FIG. 6A-FIG. 6E show NPCs growing in different concentrations of fibrin gel. FIG. 6A shows bright phase images of cell growth at different concentrations of fibrinogen (2.5, 5, 10 mg/mL,) with fixed thrombin concentration at 1 U and the cell growth at different concentrations of thrombin (1, 5, 10 U/mL,) with fixed fibrinogen concentration at 2.5 mg/mL. FIG. 6B and FIG. 6C show cell viability (percentage of live cells) of neural cells growth at different concentrations of fibrinogen and thrombin (one-way ANOVA; interaction F (4, 38)=29.25, ***P<0.001; one-way ANOVA; interaction F (2, 24)=11.32, **P<0.01; unpaired t-test; *P<0.05). FIG. 6D shows live and dead staining (green: “live”, red: “dead”) of neural cells grown in fibrin gel at 6 hours and day 7. FIG. 6E shows the viability (percentage of live cells) of neural cells at different time points (n=9 or 10).

FIG. 7A-FIG. 7B show NPCs grown in the fibrin gel. FIG. 7A shows bright phase image of NPCs grown in fibrin gel at one month. FIG. 7B shows immunostaining for neural markers TUJ1, OTX2 and MAP2 at day 4 in the gel. Hoe: Hoechst 33342.

FIG. 8A-FIG. 8F show survival and differentiation of human neural cells in fibrin hydrogel. FIG. 8A shows that neurons had pyramidal morphology and formed dendritic structures, revealed by GFP after one month of growth in the gel. FIG. 8B shows immunostaining for neuronal markers MAP2 and NeuN at day 39. FIG. 8C shows quantitative analysis of the expression of TUJ1, MAP2 and NeuN (n=10) for day 4, day 19 and 39 in fibrin gel. TUJ1, day 4 vs day 19, multiple t-test, **P<0.01; day 4 vs day 39, multiple t-test, ****P<0.0001. MAP2, day 4 vs day 19, multiple t-test, ***P<0.001; day 4 vs day 39, multiple t-test, ****P<0.0001. NeuN, day 4 vs day 19, multiple t-test, ***P<0.001; day 4 vs day 39, multiple t-test, ****P<0.0001. FIG. 8D shows immunostaining for dendritic spine marker Drebrin with MAP2 at day 40 in the gel. FIG. 8E and FIG. 8F show immunostaining for synaptic markers vGlut1, SYN1 with TUJ1 or MAP2 at day 35 in fibrin gel. Hoe: Hoechst 33342. Scale bars, 200 μm (c), 20 μm (i), 10 μm (h, j).

FIG. 9 is a schematic showing the design of the printing process disclosed herein.

FIG. 10A-FIG. 10E show the printed structure produced according to the methods disclosed herein. FIG. 10A shows the overview of the printed structure (5×2.5×0.5 mm) using the testing bio-ink in a 24-well plate. FIG. 10B shows printed neural tissue with a “green-green-red-red” layered pattern 7 days post-printing. Immunostaining of green fluorescent protein (GFP), monomeric red fluorescent protein mCherry and MAP2. FIG. 10C shows bright phase images of printed layers using hyaluronic acid and fibrin gel as bio-ink. FIG. 10D shows immunostaining for GFP⁺ layer and GFP⁻ layer with MAP2 staining at day 7 after printing. FIG. 10E shows immunostaining for SYN1 across two layers. Daf, days after growth in fibrin gel. Hoe: Hoechst 33342.

FIG. 11 shows the 3D reconstruction of printed neural tissues. The 3D image of printed neural tissue (20 days post-printing) was stained for GFP, mCherry and MAP2. The inset is enlarged with split channels on the right and 3D reconstruction view at the bottom. Hoe: Hoechst 33342.

FIG. 12A-FIG. 12B show the incorporation of GABAergic interneurons and glutamate neurons into the printed tissue. FIG. 12A is a schematic diagram illustrating the design of experiments disclosed in Example 1. FIG. 12B shows immunostaining of tissue with GABA and MAP2 3 days post-printing. Scale bar is 100 μm, Hoe: Hoechst 33342.

FIG. 13A-FIG. 13B show synapse formation of the printed tissue. FIG. 13A shows immunostaining of printed tissues with cortical and MGE progenitors for SYN1 and PSD95 puncta at day 20 after printing. FIG. 13B shows immunostaining for synaptic markers SYN1, vGAT and PSD95 with MAP2 at day 30 after printing.

FIG. 14A-FIG. 14F show electrophysiological recording of printed tissue. FIG. 14A is a schematic diagram illustrating the electrophysiological recording of the printed tissue. FIG. 14B shows spontaneous action potential (sAP) of glutamate cortical cell from printed tissue incorporated with Glut and GABA neurons at day 21. FIG. 14C shows representative traces of expanded potential stem cells (EPSCs) recorded from glutamatergic neurons in tissues with Glut neuron-only or Glut and GABA (Glut+GABA) tissues at 5 weeks post-printing. FIG. 14D shows quantitative analysis of EPSC frequency for different groups 5 weeks after printing (t-test, **P<0.01). FIG. 14E shows representative traces of IPSCs recorded from glutamatergic neurons in printed tissue with Glut neuron-only or Glut and GABA neurons (Glut+GABA) at 5 weeks post-printing. FIG. 14F shows quantitative analysis of IPSC frequency for different groups 5 weeks after printing (t-test, ****P<0.0001).

FIG. 15A-FIG. 15B show the maturation of neurons in printed tissues. FIG. 15A shows the immunostaining of printed tissue for GFAP, NeuN and MAP2 at day 30 after printing. FIG. 15B shows the quantitative analysis of NeuN expression which indicated more than 90% of neurons were NeuN⁺.

FIG. 16A-FIG. 16G show incorporation of neurons and astrocytes into the printed tissue. FIG. 16A is schematic diagram illustrating the design of the experiments disclosed in Example 1. FIG. 16B shows immunostaining of tissue for GFAP and MAP2 (3 days post-printing). FIG. 16C shows immunostaining of astrocytes and neurons in printed tissue for GFAP and MAP2 at day 60 post-printing. FIG. 16D shows the 3D morphology of astrocytes with staining of GFAP in printed tissue at day 60. FIG. 16E shows the immunostaining of tissue with GLT-1 and GFAP at day 60. FIG. 16F shows the immunostaining for GFP-GCaMP6 and mCherry at day 30 post-printing. FIG. 6G shows 3D imaging from f. Hoe: Hoechst 33342. Scale bars, 100 μm (b), 10 μm (d).

FIG. 17A-FIG. 17H show calcium and glutamate live imaging of printed tissues. FIG. 17A is a schematic diagram illustrating calcium imaging of printed tissues with neurons and astrocytes. Neurons were mCherry labeled and astrocytes were infected GCaMP6 before printing. FIG. 17B shows cells in printed tissue responding to stimulation with high concentration of KCl solution. FIG. 17C shows representative traces of GCaMP6 imaging. ROI1-6 indicated GCaMP6-astrocytes and ROI7,8 indicated mCherry-neurons. ROI, region of interest. FIG. 17D shows total GCaMP6 amplitudes of ΔF/F in tissues at 2, 4, and 6 weeks after printing (one-way ANOVA, **P<0.01; *P<0.05). FIG. 17E is a schematic diagram illustrating glutamate imaging of printed tissues with neurons and astrocytes. Neurons are mCherry labeled and astrocytes are infected with iGluSnFR before printing. FIG. 17F shows cells in printed tissue responding to stimulation with high concentration KCl solution. FIG. 17G shows representative traces of iGluSnFR imaging. ROI1-5 indicated iGluSnFR-astrocytes. FIG. 17H shows total iGluSnFR amplitudes of ΔF/F in tissues at 2 and 4 weeks after printing (t-test, **P<0.01).

FIG. 18A-FIG. 18E show the application of the 3D printing in Alexander disease (AxD). FIG. 18A is a schematic diagram illustrating the design of experiments disclosed in Example 2. FIG. 18B shows immunostaining of control and AxD tissues for GFAP showing the aggregation of GFAP fibers at day 60 post-printing. White triangles indicate the GFAP aggregates. FIG. 18C shows immunostaining of control and AxD tissues for GLT-1, GFAP and MAP2 at day 20 post-printing. FIG. 18D shows immunostaining of tissue for SYN1, GFAP and MAP2 (30 days post-printing). FIG. 18E shows quantitative analysis of SYN1 puncta density (t-test, ****P<0.0001). Hoe: Hoechst 33342. Scale bars, 5 μm (b), 20 μm (d), 50 μm (c).

FIG. 19A-FIG. 19G show functional characterization in modeling Alexander disease. FIG. 19A is a schematic diagram illustrating calcium imaging of printed tissues for AxD model. Healthy neurons were mCherry labeled and AxD or control astrocytes were infected GCaMP6 before printing. FIG. 19B is a heatmap showing ΔF/F of GCaMP6 signal from control and AxD. FIG. 19C shows total GCaMP6 amplitudes of ΔF/F in control and AxD tissues overtime (multiple t-tests, *P<0.05; **P<0.01; ***P<0.001). FIG. 19D is a schematic diagram illustrating glutamate imaging of printed tissues for AxD model. Healthy neurons were mCherry labeled and AxD or control astrocytes were infected iGluSnFR before printing. FIG. 19E shows cells in printed tissue responding to stimulation with high concentration KCl solution. FIG. 19F shows representative traces of iGluSnFR change (ΔF/F) from control and AxD tissues. FIG. 19G shows total iGluSnFR amplitudes of ΔF/F in control and AxD tissues at 2 and 4 weeks after printing (multiple t-tests, *P<0.05; **P<0.01). Scale bar is 100 μm.

FIG. 20 shows the incorporation of neurons, astrocytes, and microglia in the printed tissue. Immunostaining of Iba1, GFAP and MAP2 shows the presence of neurons, astrocytes, and microglia in the printed tissue 3 days after printing.

FIG. 21 shows photomicrographs showing the immunofluorescence markers DARPP32, GABA and GAD67 from differentiated striatal progenitors produced from human PSCs.

FIG. 22A-FIG. 22B are photomicrographs illustrating characterization of the printed cortical-striatal tissues. FIG. 22A show representative images of printed cortical-striatal tissues for GFP, mCherry and DARPP32 staining 5 days after printing, whereas FIG. 22B shows immunostaining of printed tissues for GFP, mCherry and MAP2 at 2 weeks after printing.

FIG. 23A-FIG. 23C illustrates planning and execution of printing cortical-striatal tissue. FIG. 23A a is a schematic diagram illustrating experimental design for printing cortical-striatal tissue. FIG. 23B shows immunostaining of tissue for GFP and mCherry to show the presence of printed cortical-striatal layers at 15 days post-printing (Scale bar, 100 μm). FIG. 23C shows the presence of neurites that cross the cortical-striatal layer boundary; arrowhead indicates contact between the GFP⁺ cortical neurite and mCherry⁺ striatal neuron at 21 days after printing. Hoe, Hoechst 33342.

FIG. 24A-FIG. 24E show calcium imaging of printed cortical-striatal tissue. FIG. 24A is a schematic diagram illustrating calcium imaging of printed tissues with cortical and striatal neurons. Cortical neurons were ChR2 cells, and striatal neurons were non-colored and infected with lentivirus-jRGECO1b before printing. FIG. 24B is a photomicrograph of printed tissue (2 weeks post-printing) responding to light stimulation, showing changes in jRGECO1b signals (red) before and after stimulation. FIG. 24 C shows representative traces of calcium imaging from two regions of interest. ROI, region of interest. FIG. 24D shows total traces of calcium imaging obtained in these experiments, FIG. 24E shows total jRGECO1b amplitudes of ΔF/F in tissues after light-stimulation (21 samples from five different batches).

FIG. 25A-FIG. 25F showfunctional characterization of printed cortical-striatal tissue. FIG. 25A is a schematic diagram illustrating whole-cell patch-clamp recording with optogenetic activation in printed cortical-striatal tissues. Cortical neurons were ChR2 cells, and striatal neurons were mCherry labeled. FIG. 25B-25D are representative electrophysiological traces of spontaneous action potentials, EPSPs and EPSCs of striatal neurons within printed tissues by light stimulation. FIG. 25E shows the percentage of responsive cells with light stimulation for EPSCs recording. FIG. 25F is a bar graph showing quantitative analysis of EPSCs frequency before, during and after light stimulation (paired t-test, *P<0.05; Data are mean±s.e.m. (neurons from three different batches).

FIG. 26 shows still frames from video recordings showing contraction of printed cortical-muscle tissue. Time-lapse of muscle cell contraction in printed muscle tissue at day 30 after printing. Red arrows indicate muscle cell movement.

### DETAILED DESCRIPTION

The disclosure generally relates to methods and compositions for preparing a neural tissue construct. In particular, provided herein are methods for generating a neural tissue construct using glutamatergic cortical progenitor cells; GABAergic interneuron progenitor cells; and bio-ink. These neural tissue constructs are useful for understanding the wiring of neural networks, modeling pathological processes, and serving as platforms for drug testing.

All publications, including but not limited to patents and patent applications, cited in this specification are herein incorporated by reference as though set forth in their entirety in the present application.

For the purposes of promoting an understanding of the principles of the disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

As used in the specification, articles “a” and “an” are used herein to refer to one or to more than one (i.e., at least one) of the grammatical object of the article. By way of example, “an element” means at least one element and can include more than one element.

“About” is used to provide flexibility to a numerical range endpoint by providing that a given value can be “slightly above” or “slightly below” the endpoint without affecting the desired result. The term “about” in association with a numerical value means that the numerical value can vary by plus or minus 5% or less of the numerical value.

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements, or steps but not the exclusion of any other integer or step or group of integers or steps.

As used herein, “and/or” refers to and encompasses any and all possible combinations of one or more of the associated listed items, as well as the lack of combinations where interpreted in the alternative (“or”).

Recitation of ranges of values herein are merely intended to serve as a succinct method of referring individually to each separate value falling within the range, unless otherwise indicated herein. Furthermore, each separate value is incorporated into the specification as if it were individually recited herein. For example, if a range is stated as 1 to 50, it is intended that values such as 2 to 4, 10 to 30, or 1 to 3, etc., are expressly enumerated in this disclosure. These are only examples of what is specifically intended, and all possible combinations of numerical values between and including the lowest value and the highest value enumerated are to be considered to be expressly stated in this disclosure.

The term “contacting” includes the physical contact of at least one substance to another sub stance.

Unless otherwise defined, all technical terms used herein have the same meaning as commonly understood by a person of ordinary skill in the art to which this disclosure belongs.

In particular embodiments provided herein is a method of generating a neural tissue construct, comprising:

(a) horizontally depositing by bioprinting on a suitable surface a band comprising a mixture, wherein the mixture comprises

(i) glutamatergic cortical progenitor cells;

(ii) GABAergic interneuron progenitor cells; and

(iii) bio-ink

(b) repeating the depositing step of (a) a plurality of times to form a plurality of bands; and

(c) maturing the deposited plurality of bands to allow the cells to form a neural tissue construct.

As used herein, “bio-ink” means a liquid, semi-solid, or solid composition for use in bioprinting. In some embodiments, the bio-ink comprises an extrusion compound (i.e., a compound that modifies the extrusion properties of the bio-ink). Examples of extrusion compounds include, but are not limited to gels, hydrogels, peptide hydrogels, amino acid-based gels, surfactant polyols (e.g., Pluronic F-127 or PF-127), thermo-responsive polymers, hyaluronates, alginates, extracellular matrix components (and derivatives thereof), collagens, gelatin, other biocompatible natural or synthetic polymers, nanofibers, and self-assembling nanofibers.

In some embodiments, the bio-ink comprises a hydrogel and hyaluronic acid. Suitable hydrogels include those derived from fibrinogen, collagen, hyaluronate, hyaluronan, fibrin, thrombin, alginate, agarose, chitosan, and combinations thereof. In some embodiments, the hydrogel comprises fibrinogen and thrombin. In particular embodiments, the hydrogel comprises 2.5 mg/mL fibrinogen and 0.5 U thrombin.

In some embodiments, the hydrogel further comprises a protease inhibitor such as a serine inhibitor. In particular embodiments, the protease inhibitor is aprotinin, which inhibits several serine proteases, specifically trypsin, chymotrypsin, and plasmin.

In particular embodiments, the progenitor cells are mixed with the hydrogel and hyaluronic acid at a density of about 1×10⁶/mL, about 1×10⁷/mL, about 1×10⁸/mL, or about 1×10⁹/mL.

In some embodiments, the hydrogel comprises components that are crosslinkable. For example, in some embodiments, suitable hydrogels include fibrinogen-containing crosslinkable hydrogels. In various embodiments, suitable hydrogels comprise about 0.1, 0.5, 1, 2, 2.5, 3, 3.5, 4, 4.5, 5, 5.5, 6, 6.5, 7, 7.5, 8, 8.5, 9, 9.5, 10, or more percent fibrinogen. In various embodiments, suitable hydrogels comprise about 15, 20, 25, 30, 35, 40, 45, 50 or more percent fibrinogen. In some embodiments, following bioprinting, constructs are incubated with an agent to chemically crosslink the hydrogel, such as a solution comprising CaCl₂, thrombin and transglutaminase (TG).

As used herein, “bioprinting” means utilizing precise deposition of the mixture comprising the progenitor cells and bio-ink via methodology that is compatible with an automated or semi-automated, computer-aided, three-dimensional prototyping device (e.g., a bioprinter).

In some embodiments, a plurality of bands are bioprinted next to each other on a suitable surface to form a neural tissue construct. In various embodiments, 2, 3, 4, 5, 6, 7, 8, 9, 10, 11, 12, 13, 14, 15, or more layers are bioprinted to form a neural tissue construct.

In particular embodiments, the bands are printed horizontally one layer at a time at the dimension of 5000 (L)×500 (W)×50 (H) Typically, the bands of mixture have a horizontal thickness of about 100 μm or less, about 75 μm or less, about 50 μm cm or less. In particular embodiments, the bands have a thickness horizontally of about 50 μm.

As used herein “suitable surface” refers to a surface which supports neural progenitor cells attachment, migration, proliferation, and/or maturation.

In particular embodiments, the plurality of bands is physically separated. To prevent the mix of the printed layers, a crosslinking agent such as for example thrombin can be added immediately following the deposit of the mixture to form the desired shape before printing the next layer.

In particular embodiments, the mixture used for bioprinting comprises glutamatergic cortical progenitor cells, GABAergic interneuron progenitor cells, and bio-ink. In particular embodiments, the mixture further comprises astrocyte progenitor cells. In particular embodiments, the mixture further comprises microglial cells.

In some cases, the progenitor cells for use in the methods provided herein are obtained by directed differentiation of human pluripotent stem cells (hPSCs). Pluripotent stem cells, including embryonic stem cells (ESCs) and induced pluripotent stem cells (iPSCs) can be used for producing the glutamatergic cortical progenitor cells, GABAergic interneuron progenitor cells and astrocyte progenitor cells.

As used herein, the term “pluripotent stem cell” (hPSC) means a cell capable of continued self-renewal and of capable, under appropriate conditions, of differentiating into cells of all three germ layers. hPSCs exhibit a gene expression profile that includes SOX2⁺ and OCT4⁺. Examples of human PSCs (hPSCs) include human embryonic stem cells (hESCs) and human induced pluripotent stem cells (hiPSCs). ESCs are commercially available from sources such as WiCell Research Institute (Madison, Wis.). As used herein, “induced pluripotent stem cells” or “iPS cells” mean a pluripotent cell or population of pluripotent cells that may vary with respect to their differentiated somatic cell of origin, that may vary with respect to a specific set of potency-determining factors and that may vary with respect to culture conditions used to isolate them, but nonetheless are substantially genetically identical to their respective differentiated somatic cell of origin and display characteristics similar to higher potency cells, such as ESCs, as described herein. See, e.g., Yu et al., Science 318:1917-1920 (2007).

The neural progenitor cells used herein may be from different stages. In particular embodiments, glutamatergic cortical neural progenitor cells are 21-day human pluripotent stem cells (hPSCs)-derived glutamatergic cortical neural progenitor cells. In particular embodiments, the GABAergic interneuron progenitor cells are 21-day human pluripotent stem cells (hPSCs)-derived GABAergic interneuron progenitor cells.

In particular embodiments, the glutamatergic cortical progenitor cells and the GABAergic interneuron progenitor cells are present in the mixture at a ratio of 4:1. In particular embodiments, the glutamatergic cortical progenitor cells, the GABAergic interneuron progenitor cells, and the astrocyte progenitor cells are present in the mixture at a ratio of 5:1:4.

Maturing the deposited plurality of bands to allow the cells to form a neural tissue construct can take place in any suitable media. Suitable media include, for example, a neural basal medium. In particular embodiments, the neural tissue construct is cultured for a sufficient time to form a functional neural network and/or a functional neural-glia network, for example, at least 7 days, at least 14 days, at least 3 weeks, or at least 4 weeks in culture. In particular embodiments, the neuronal basal medium comprises at least one of 2% B-27, a Brain Derived Neurotrophic Factor (BDNF), a Glial cell line-derived neurotrophic factor (GDNF), ROCK inhibitor, or γ-secretase and notch pathway inhibitor.

Functional assessment of the printed neural constructs can include electrophysiological recording such as whole-cell patch clamping. A functional neural tissue construct will have for example inward Na⁺ and outward K⁺ currents, spontaneous action potentials (sAP) after printing, and excitatory and inhibitory postsynaptic currents. A functional neural tissue construct comprising astrocytes will have calcium flux and glutamate uptake in astrocytes in response to neuronal excitation.

Also provided herein is a neural tissue construct produced by the methods disclosed herein.

In particular embodiments disclosed herein are methods of screening a test agent, the method comprising: depositing a test agent by bioprinting on the neural tissue construct disclosed herein; measuring a functional parameter of the contacted neural tissue construct; and comparing the functional parameter to that parameter measured in neural tissue construct which has not been contacted with the test agent, wherein modulation of the functional parameter after contact with the test agent indicates the test agent is a candidate therapeutic agent.

In some embodiments, a test agent may be characterized as having neural toxicity when the test agent modulates the functional parameter away from physiologically acceptable conditions.

In some embodiments, the cells of the neural tissue construct express a detectable marker that is responsive to the test agent. Examples of such detectable markers include but are not limited to green fluorescent protein (GFP), mCherry, and ChR2-EYFP (see, Dong et al., 2020, iScience 23: 100829) and red calcium indicator jRGECO1b (Dana et al., 2016, Elife 5).

“Test agent” refers to a molecule assessed for its ability to alter a specific phenotypic endpoint. Examples of test agents include, but are not limited to, (i) organic compounds of molecular weight less than about 600 daltons; (ii) nucleic acids; (iii) peptides (including stapled peptides); (iv) polypeptides; and (v) antibodies or antigen-binding fragments thereof.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

Without limiting the disclosure, a number of embodiments of the disclosure are described below for purpose of illustration.

### EXAMPLES

The Examples that follow are illustrative of specific embodiments of the disclosure, and various uses thereof. They are set forth for explanatory purposes only and should not be construed as limiting the scope of the disclosure in any way.

### Example 1: Generation of Neural Tissue Construct

Neuronal progenitors and astrocytes progenitors were generated from human pluripotent stem cells (human PSCs), including human embryonic stem cells (ESCs) and human induced pluripotent stem cells (iPSCs). The progenitors differentiated from human PSCs were analyzed and characterized by immunofluorescence.

First, human ESCs (H9, GFP-H9, mCherry-H9) were maintained on mouse embryonic fibroblast (MEF) feeder in a stem cell growth medium or on Matrigel-coated plates in TeSR-E8 medium (StemCell Technologies, Inc., Vancouver, Canada) as described previously (Li, X. et al. Stem Cell Reports 11, 998-1008, doi:10.1016/j.stemcr.2018.08.019 (2018); Yan, Y. et al. Biomaterials 73, 231-242, doi:10.1016/j.biomaterials.2015.09.020 (2015)). For MEF feeder-based culture, cells were passaged weekly by using dispase (1 mg/mL, Gibco) and plating on a monolayer of irradiated MEF (WiCell). The hPSC culture medium consisted of DMEM/F12 basal medium (Gibco), 20% KnockOut serum replacement (Gibco), 0.1 mM b-mercaptoethanol (Sigma), 1 mM L-glutamine (Gibco), nonessential amino acids (Gibco), and 4 ng/mL fibroblast growth factor (FGF)-2 (R&D Systems). For TeSR-E8 medium-based culture, cells were passaged every 6-7 days by accutase (Chemicon) and plated on Matrigel-coated 6-well plate for monolayer culture in the presence of ROCK inhibitor Y27632 (10 μm, Sigma) to promote cell survival.

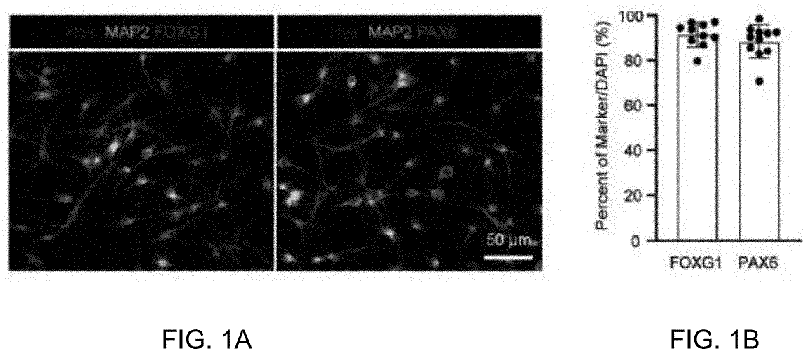

The generation of cortical neural progenitors was performed as previously described (Yan, Y. et al. Biomater 42, 114-126, doi:10.1016/j.actbio.2016.06.027 (2016); Chambers, S. M. et al. Nat Biotechnol 27, 275-280, doi:10.1038/nbt.1529 (2009); Yan, Y. et al. Tissue Eng Part A, doi:10.1089/ten.TEA.2017.0423 (2018)). Briefly, hPSCs were seeded into Ultra-Low Attachment (ULA) 24-well plates (Corning, Inc., Corning, N.Y.) at 3.0-3.5×10⁵ cells per well in 1 mL of TeSRE8 medium and grown for 2 days. ROCK inhibitor Y27632 (10 μm) was added during the seeding and removed after 24 h. Then, the culture was switched to neural differentiation medium composed of Dulbecco's modified Eagle's medium/nutrient mixture F-12 (DMEM/F12) plus 2% B27 minus vitamin A serum-free supplement (Life Technologies). At day 1 in neural medium, the cells were treated with dual SMAD signaling inhibitors: 10 μM SB431542 (Sigma) and 100nM LDN193189 (Sigma). After 7 days, the cells were incubated with cyclopamine (1 μM, Sigma), FGF-2 (10 ng/mL, R&D System) and epidermal growth factor (EGF, 10 ng/mL, R&D System) for another 8 days. The cells cultures were maintained in FGF-2 until day 21 in suspension culture and were dissociated by accutase for printing. The cortical progenitors at day 21 were characterized by the staining of FOXG1, PAX6 and MAP2 as seen in FIG. 1.

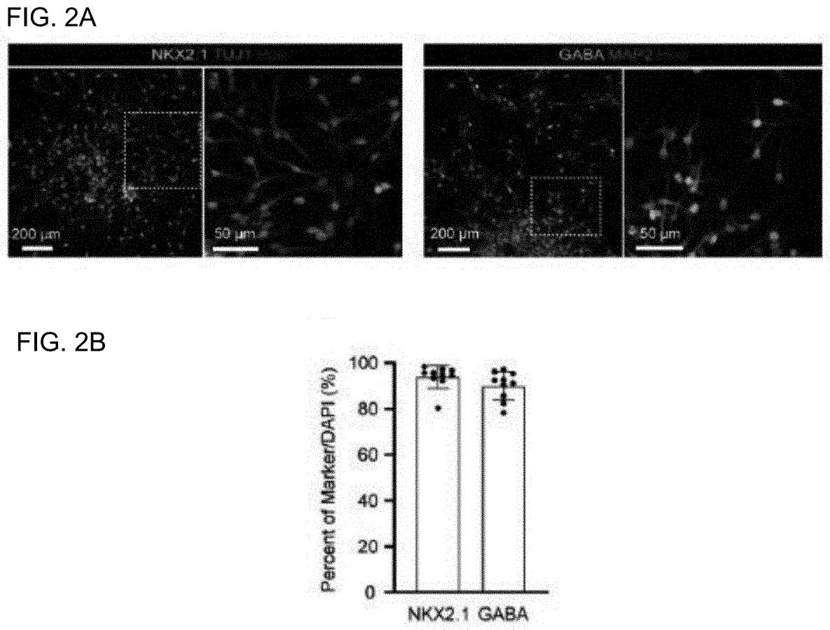

The differentiation of GABAergic interneurons was based on the previously developed protocol (Liu, Y. etal. Nat Protoc 8, 1670-1679, doi:10.1038/nprot.2013.106 (2013); Liu, Y. et al. Nat Biotechnol 31, 440-447, doi:10.1038/nbt.2565 (2013)). After 7 days of the above neural differentiation, the sonic hedgehog (SHH) activator purmorphamine (1 μM, Sigma) was added. At day 21, the GABAergic interneuron progenitors were also dissociated to single cells for printing. The GABAergic interneuron progenitors at day 21 were characterized by the staining of NKX2.1, GABA, TUJ1 and MAP2 as seen in FIG. 2.

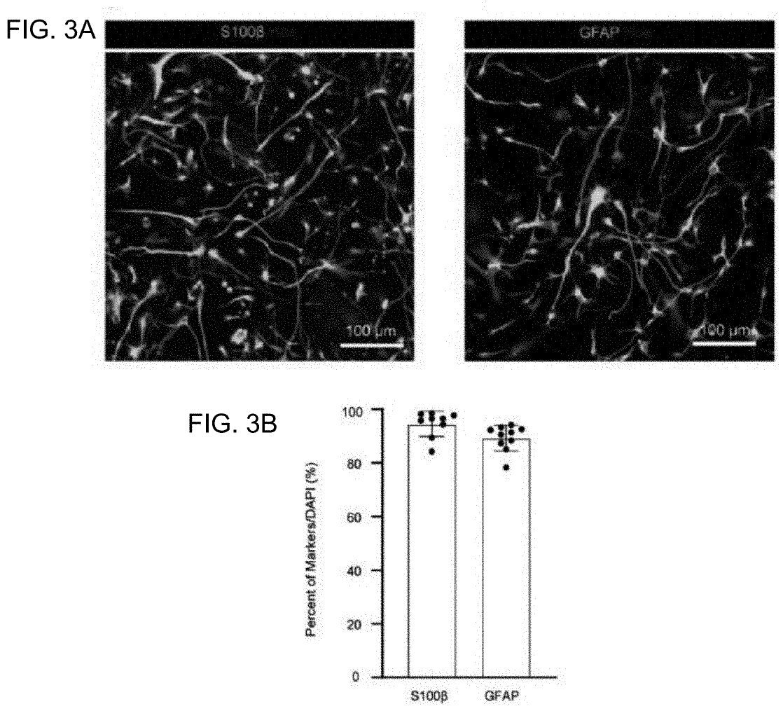

The generation of astrocyte progenitors was performed from as previously described (Li, X. et al. Stem Cell Reports 11, 998-1008, doi:10.1016/j.stemcr.2018.08.019 (2018); Krencik, etal. Nat Biotechnol 29, 528-534, doi:10.1038/nbt.1877 (2011)). The above day 21 neural spheres were dissociated with accutase to single cells, attached with a laminin substrate in the presence of CNTF (10 ng/ml, R&D System), LIF (10 ng/ml, Millipore), or FBS (FBS, 10%, Gibco). And single astrocyte progenitors were prepared for printing. The astrocyte progenitors were characterized by the staining of S1000 and GFAP as seen in FIG. 3.

The differentiated cells were characterized and analyzed by immunofluorescence. Briefly, cells on coverslips/wells were rinsed with PBS and fixed in 4% paraformaldehyde for 20 min. After rinsing with PBS twice, cells were treated with 0.3% Triton for 10 min followed by 10% donkey serum for 1 hour before incubating with primary antibodies overnight at 4° C. Cells were then incubated for 1 h at room temperature with secondary antibodies (Life Technologies). The nuclei were stained with Hoechst (Ho) (Sigma-Aldrich). Images were taken with a Nikon A1R-Si laser-scanning confocal microscope (Nikon, Tokyo, Japan). The primary antibodies used included the followings: Rabbit anti-FOXG1 (1:100, ab18259, Abcam), Mouse anti-PAX6 (1:5000, PAX6, DSHB), Rabbit anti-TUJ1 (1:10000, PBR-435P, Covance), Mouse anti-TUJ1 (1:1000, ab117988, Abeam), Rabbit anti-MAP2 (1:1000, Millipore), Mouse anti-MAP2 (1:1000, M1406-2ML Sigma), Chicken anti-MAP2 (1:5000, ab5392, Abeam), Mouse anti-NeuN (1:500, MAB377, Millipore), Rabbit anti-Drebrin (1:1000, AB10140, Millipore), Rabbit anti-vGlut1 (1:1000, 135303, Synaptic Systems), Rabbit anti-SYN1 (1:1000, 106-003, Synaptic Systems), Mouse anti-PSD95 (1:500, 124011, Synaptic Systems), Mouse anti-Gephyrin (1:500, 147011, Synaptic Systems), Rabbit anti-vGAT (1:1000, 131002, Synaptic Systems), Rabbit anti-GABA (1:1000, A2052, Sigma), Rabbit anti-GFAP (1:1000, Z033429; Dako), Mouse anti-GFAP (1:1000, IF03L, Millipore), Mouse anti-5100(3 (1:1000, ab11178, Abeam), Mouse anti-GLT1 (1:500, 611654, BD Transduction Laboratories), Goat anti-OTX2 (1:1000, AF1979, R&D), Mouse anti-NKX2.1 (1:500, MAB5460, Millipore); Rabbit anti-50X2 (1:500, AB5603, Millipore), Rabbit anti-TBR1 (1:1000, ab31940, Abeam), Rat anti-CTIP2 (1:1000, ab18465, Abeam), Mouse anti-SATB2 (1:25, ab51502, Abeam), Rabbit anti-calretinin (1:1000, 2624-1, Epitomics Inc.), Rabbit anti-calbindin (1:1000, ab25085, Abeam), Mouse anti-parvalbumin (1:500, MAB1572, Millipore), Rat anti-somatostatin (1:200, MAB354, Millipore). Multiple fields were chosen randomly under the fluorescent filter for nuclear staining throughout the coverslips in areas which contained a similar density of Hoechst+cells and the total cells were counted. The fluorescent filters were shifted during imaging to count the cells labeled by different antibodies in the same field in the same manner. The quantitative data were repeated three times.

An optimal concentration of fibrinogen and thrombin was identified by measuring the survival of hPSC-derived cortical neural progenitor cells (NPCs). Fibrin gel was prepared from fibrinogen and thrombin. Stock solution of 50 mg/mL fibrinogen, 100 U thrombin, 250 mM CaCl2 (100×) and 10 mg/mL aprotinin (20×) were prepared in the following manner: Fibrinogen (F3879, Sigma) was dissolved in Dulbecco's phosphate buffered saline (DPBS) without calcium and magnesium for 4 h at 37° C. The solution was sterile filtered and stored at −80° C. for use. CaCl (Sigma) was dissolved in deionized (DI) water and filtered for use. Thrombin (T7009, Signa) was dissolved in DPBS and sterile filtered. The solution was stored at −20° C. until use. Aprotinin (A1153, Sigma) was dissolved in DPBS and stored at −20° C. until use.

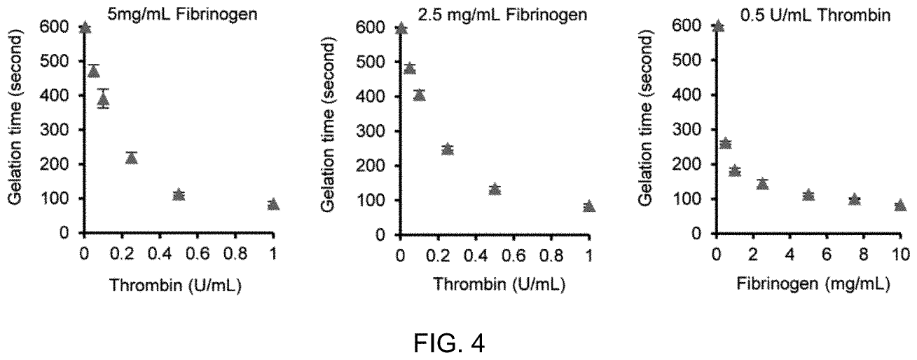

The gelation testing of the fibrin gel was modified from previously reports (Kubota, K. et al. Colloids Surf B Biointerfaces 38, 103-109, doi:10.1016/j.colsurfb.2004.02.017 (2004); Sproul, E. P., Hannan, R. T. & Brown, A. C. Methods Mol Biol 1758, 85-99, doi:10.1007/978 4939-7741-3_7 (2018)). Different concentrations of fibrinogen solutions and thrombin solutions were prepared. 1 μL fibrinogen solution was dropped onto cover slide and was mixed well with the same volume of thrombin. To test the gelation, a pipette tip of 2 μL was touched the gel to check whether the gel was solid or not (no liquid could be taken by pipette tip). The time from the adding of thrombin and formation of gel was recorded and defined as the gelation time. The gelation time of different composition of fibrinogen and thrombin are shown in FIG. 4. At a constant concentration of fibrinogen (5 or 2.5 mg/mL), the gelation time decreased with an increased level of thrombin. At 0.5 U thrombin, the gelation time was also reduced with increasing fibrinogen concentrations. 2.5 mg/mL fibrinogen and 0.5 U thrombin was used for hydrogel construction, yielding a gelation time of about 145±10 seconds which was sufficient to print a 24-well plate.

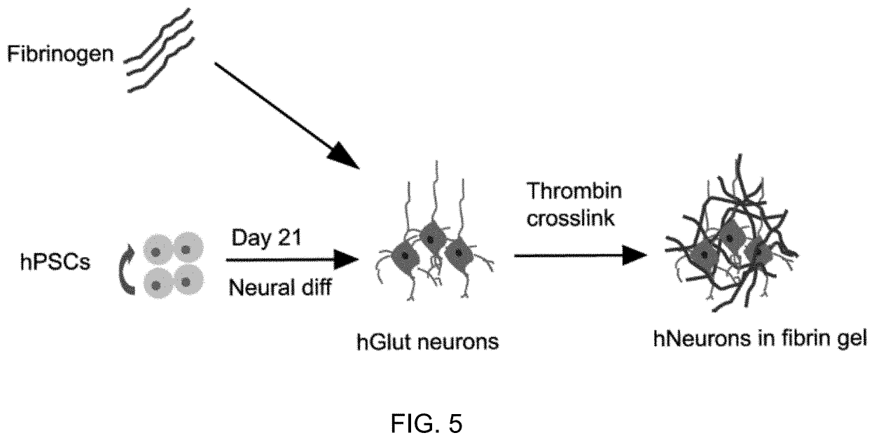

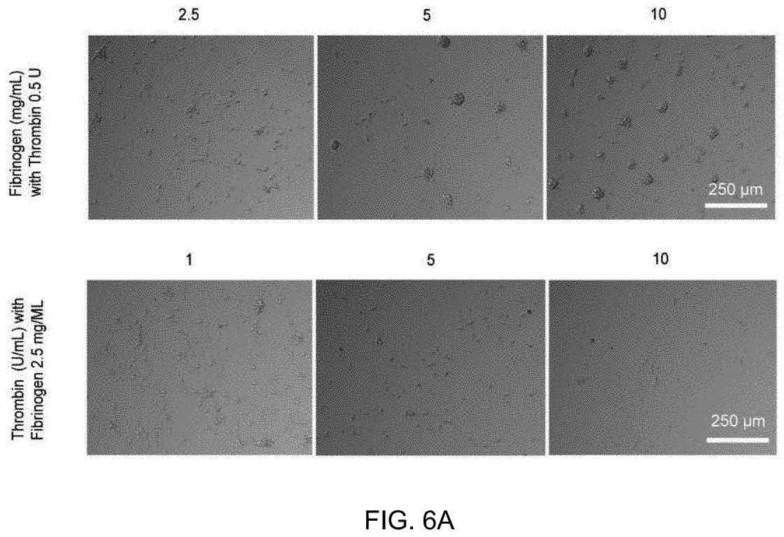

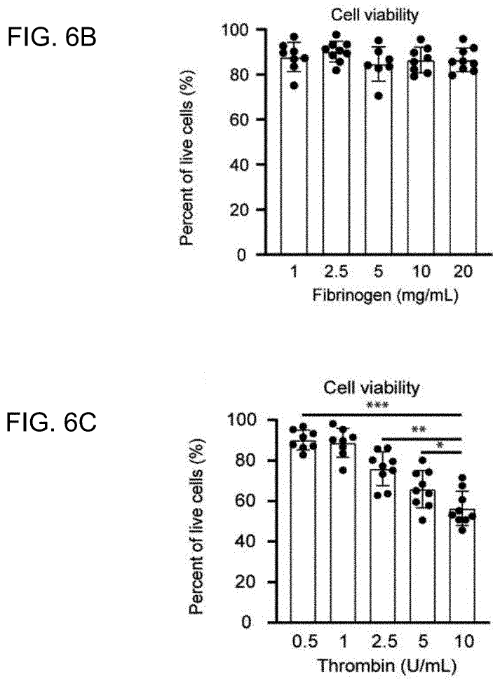

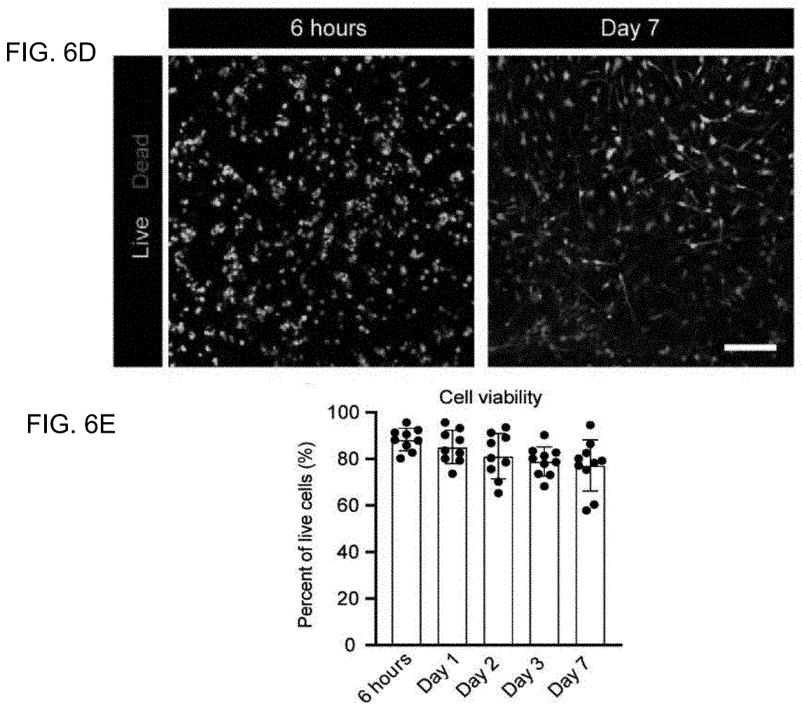

For cell culturing in the fibrin gel, dissociated hPSC-NPCs at a density of 1×10⁶ were mixed with fibrinogen at the following concentrations (1, 2.5, 5, 10 and 20 mg/mL) as show in FIG. 5. 0.5 mg/mL aprotinin was added for preventing gel degradation. 1 μL of the fibrinogen-laden cells were dropped on the poly-O coated plates at RT (room temperature). 1 different concentrations of thrombin (0.5, 1, 2.5, 5 and 10 U) solution with 2.5 mM CaCl₂ were then added and mixed with the cells-fibrinogen solution. Fresh medium was added after gelation. Cells were encapsulated in the fibrin gel constructs and cultured at 37° C. incubator for characterization. The cell viability, indicated by percent of live cells to total cells from Live/Dead assay, decreased with increasing thrombin concentrations at a fixed fibrinogen concentration of 2.5 mg/ml, while it was not influenced by the increasing concentrations of fibrinogen at the fixed low concentration of thrombin (0.5 U) as shown in FIG. 6. The cells, however, tended to aggregate at higher fibrinogen levels. Thus, 2.5 or 5 mg/mL fibrinogen with 0.5 or 1 U thrombin was used for identifying an optimal gelation time.

The cell viability was measured by Live/Dead assay. Briefly, Live/Dead® staining kit (Molecular Probes) was used to assess cell viability. Immediately after harvesting, the cells were incubated in DMEM/F12 containing 1 μM calcein AM and 2 μM ethidium homodimer I for 30 min. Cells were washed and representative images were taken of samples. The number of live (green) and dead (red) cells were counted in each field using Cell Counter in Image-J. The live/dead cell numbers from the five images of one sample were averaged to give each data point and five samples were used to determine the viability.

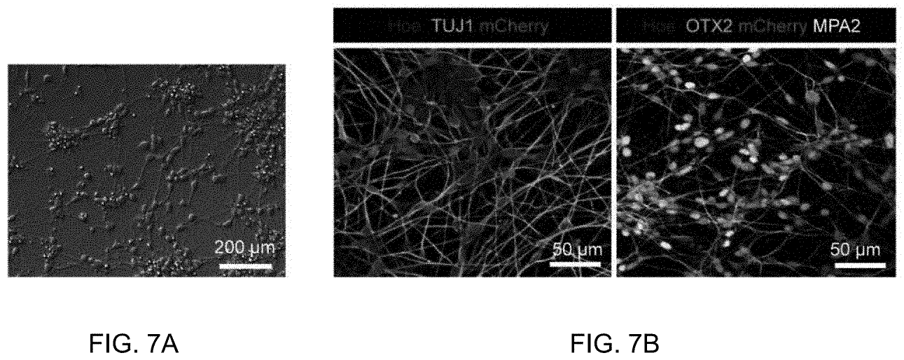

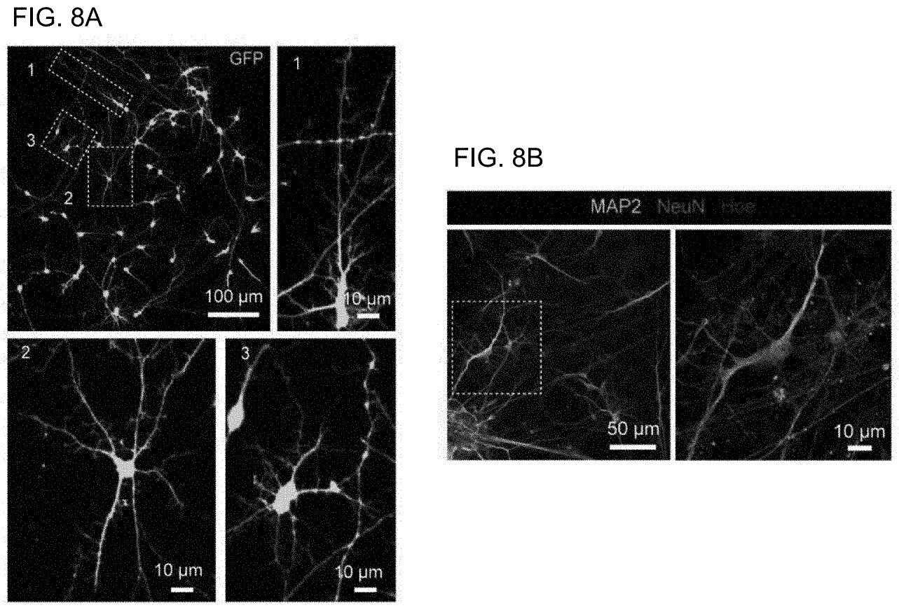

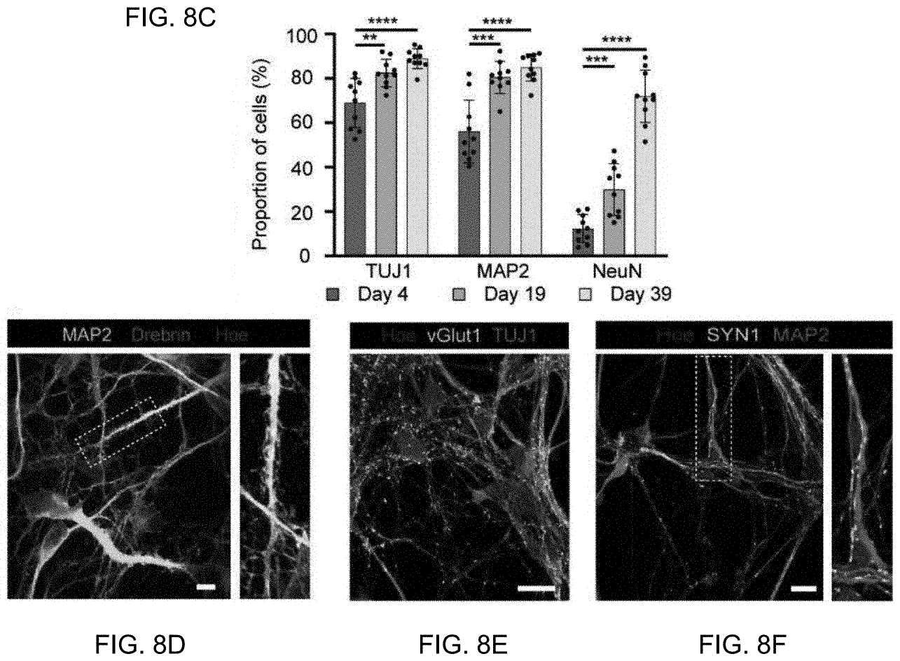

A special requirement for neural tissue printing is for the bio-ink to support neurite growth and synaptogenesis. The cortical NPCs, differentiated from GFP- or mCherry-labeled hESCs for 21 days, expressed forebrain cortical progenitor markers FOXG1 and PAX6 as shown in FIG. 1. When cultured in the fibrin gel, the progenitors became process-bearing, TUJ1 positive neurons as early as at day 4 and progressively expressed mature neuronal markers MAP2 and NeuN as shown in FIG. 7 and FIG. 8. Many neurons displayed a pyramidal morphology after 1 month of culture in the gel. Importantly, the neurons extended elaborate neurites with fine dendritic structures, revealed by GFP labeling. This was confirmed by positive staining for drebrin, a dendritic spine marker at day 40. Synaptic puncta, identified by staining for vGlut1 and synapsin (SYN1), were observed on TUJ1⁺ or MAP2⁺ neurites at day 35. Collectively, the fibrin gel supported the maturation and synaptogenesis of the cortical neurons.

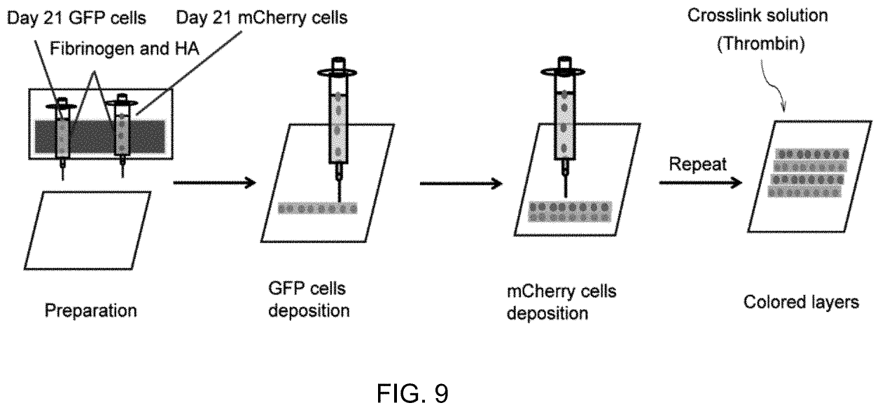

After proving that the fibrin supported neural cells growth, maturation and functions, the cells were printed to fabricate patterned structure. The brain is organized in layers or nuclei which are connected to each other by synapses. It was thus explored to construct a layered neural tissue in which the printed neural progenitors mature and form synapses across layers while the structure was maintained. To avoid the need of sectioning the “tissue block” to reveal the layers and to facilitate direct, live, microscopic observation, the cell layer, or “band” of ˜50 um thickness was printed horizontally, one after another. These horizontal “bands”, when turned 90°, exhibit vertical “layers” as shown in FIG. 9.

The printing procedure was performed as shown in FIG. 9. Briefly, the printers were from CELLINK®, INKREDIBLE+™ and BioX™ (https://www.cellink.com/bioprinting/bio-x/). The printers had two or three printheads, which could deposit multiple types of cells at the same time. The dispensing apparatuses were connected to the printer to extrude the different bio-inks through the micronozzle with different inner diameters. Before printing, the UV light was turned on for 5 min to make sterile environment for printing. For the printing with cell-laden bio-inks, printing speed of 5 mm/s was used (line-dispensing printing mode) and printing pressure of 50 kPa using the blunt needle of 30 G (NZ5300505001, Cellink, inner diameter 150 μm) or blunt needle of 27 G (NZ6270505001, Cellink, inner diameter 200 μm). All the print paths were controlled using G-code commands, which were generated by the software Slic3R from 3 models.

The bio-ink for printing was prepared from hyaluronic acid (HA) and fibrin gel. Briefly, bio-ink was made of HA (53747, Sigma) and 10 mg/mL fibrinogen with a volume ratio of 1:1. 3% (w/v) HA was prepared in DPBS at 37° C. and stirred until completely dissolved. The printing was performed at RT. After printing, the hydrogel was crosslinked with a thrombin+transglutaminase (TG)+CaCl₂ for 3-5 min at RT. And fresh medium was added after crosslinking at 37° C. incubator for characterizations.

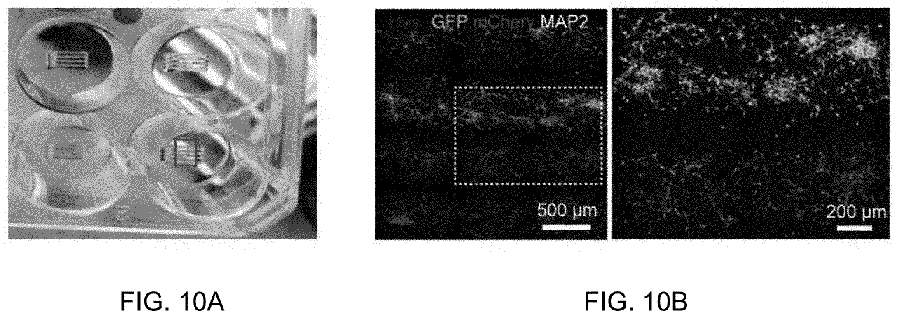

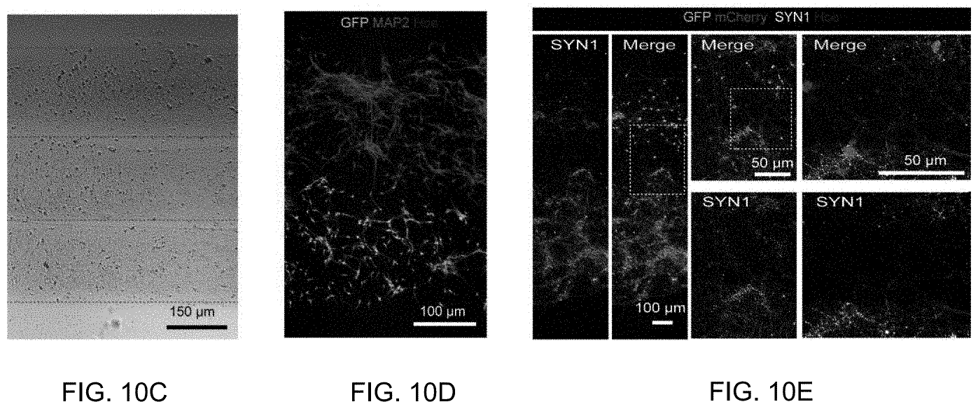

The GFP- or mCherry-labeled, hPSC-derived NPCs were dissociated in the bio-ink and printed one layer at a time at the dimension of 5000 (L)×500 (W)×50 (H) μm in one well of a 24-well plate as shown in FIG. 10. To prevent the mix of the printed layers, the crosslink agent-thrombin was added immediately following the deposit of cell-gel mixture to form the designed shape before printing the next layer. Indeed, the GFP cells in one layer were well separated from the mCherry cells in the next at 7 days after printing. The printed neural progenitor cells, when cultured in the regular differentiation medium in the presence of compound E for 3 days that promotes cell cycle exit, became positive for MAP2, and extended neuronal processes in and across the layers. The printed cells largely stayed within the designated areas, as indicated by the alternate red and green bands. Over extended culture, the layered structure with multi-cell thickness retained while the neurites became more numerous and crossed layers with formation of synapses. 3D reconstruction revealed multiple cells in depth as shown in FIG. 11. Thus, the printed tissue retained a stable structure within which the neural progenitors mature and form neural networks.

For printing the layered tissue using colored cells (GFP and mCherry-labeled) or unlabeled cells, neural cells (glutamate neuron progenitors, GABA neuron progenitors, astrocyte progenitors) were dissociated and filtered to make single cells. They were prepared separately and laden with the HA+FN bio-ink at a density of 1×10⁷/mL. The gel-laden cells were delivered to two different nozzles and deposited onto poly-O coated coverslips. The crosslink solution was added immediately after printing. The gelation was performed at RT. Printed tissues were incubated at 37° C. in fresh neural differentiation medium for the following experiments.

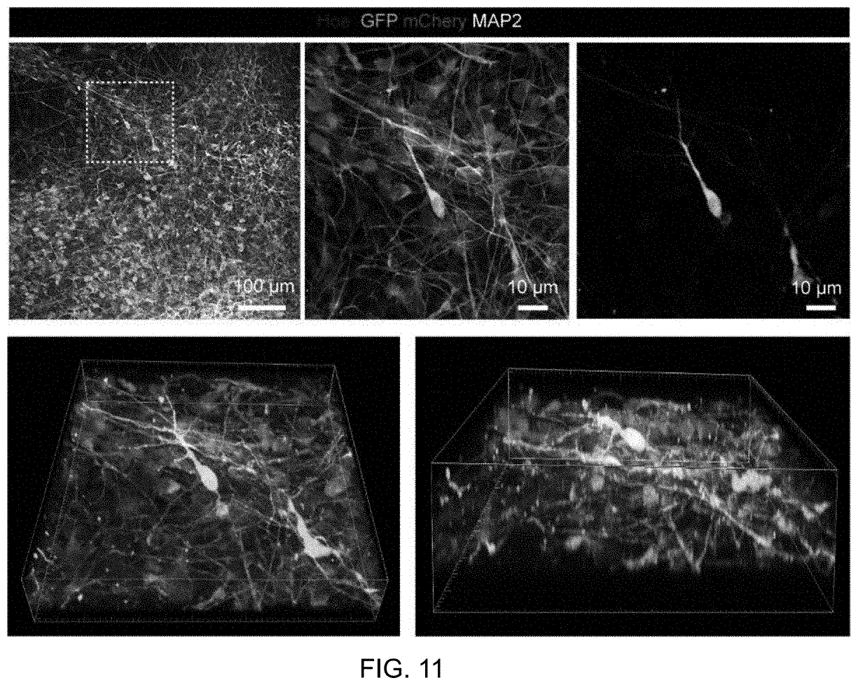

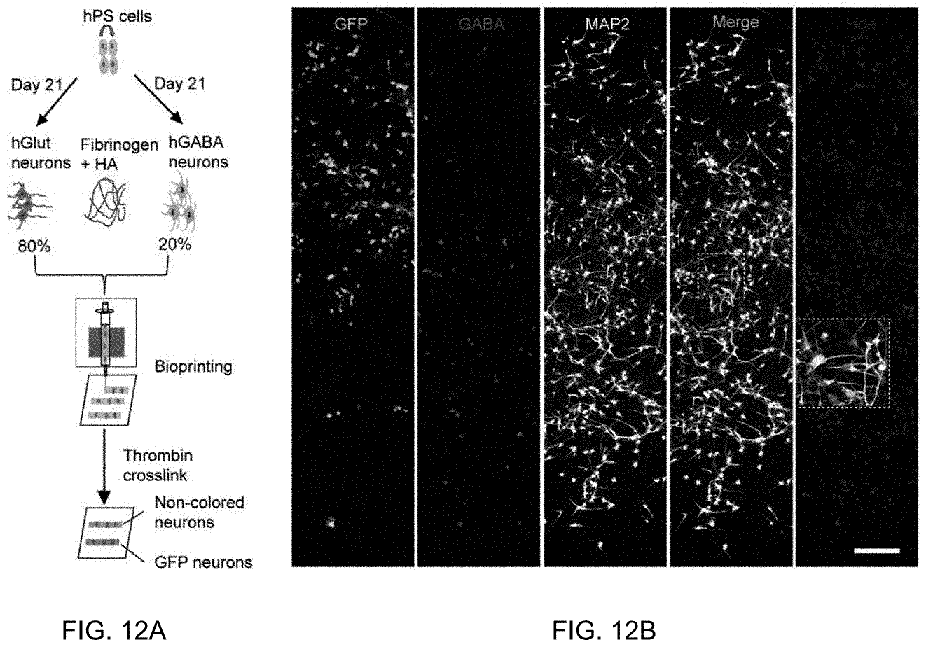

The brain functions through interactions between different neuronal types. In the cerebral cortex, two major neuronal types, GABA interneurons and glutamate neurons, synapse and interact each other. To determine if GABA interneurons and glutamate neurons, incorporated into the printed tissues, form functional synapses, MGE (GABA) and cortical (glutamate) progenitors from GFP⁺ and GFP⁻ hPSCs were generated and mixed the two progenitor populations at a ratio of 1:4 to mimic the ratio of interneurons and cortical projection neurons in the cerebral cortex before printing^{18,19} as shown in FIG. 12. For a better visualization, a two-layered tissue was printed, in which one layer was GFP-cells and the another was non-colored cells, and each layer contained the same proportion of interneuron and cortical projection neurons. The printed tissue with only cortical projection neurons was served as a reference. Both the GFP⁺ and GFP⁻ layers displayed an even distribution of GABA⁺ and neurons and MAP2⁺ and total neurons.

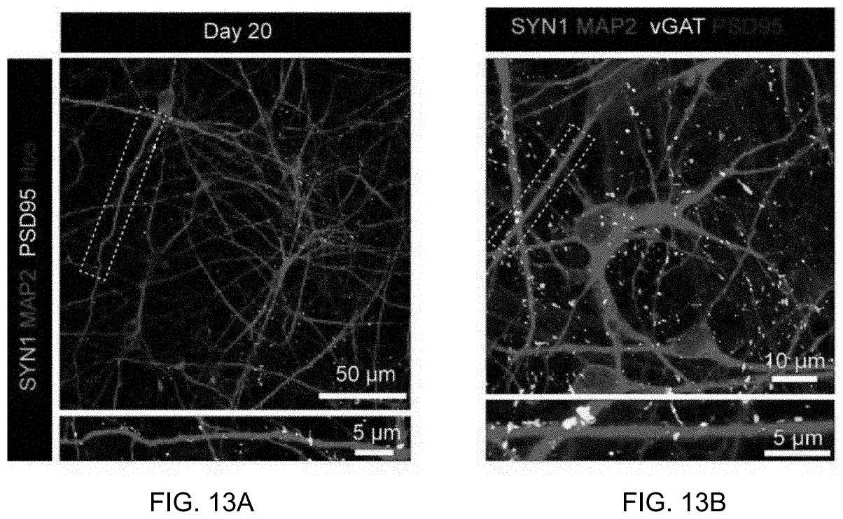

The most challenging aspect of neural tissue printing is formation of functional networks. Co-expression of the pre- and post-synaptic markers, SYN1 and PSD95 was observed, as early as day 20 after printing, indicating the formation of synapses, as shown in FIG. 13. The GABAergic neurons also formed synapses as indicated by the expression of inhibitory presynaptic protein vGAT. These observations suggest the formation of excitatory and inhibitory synapses the printed neural tissues.

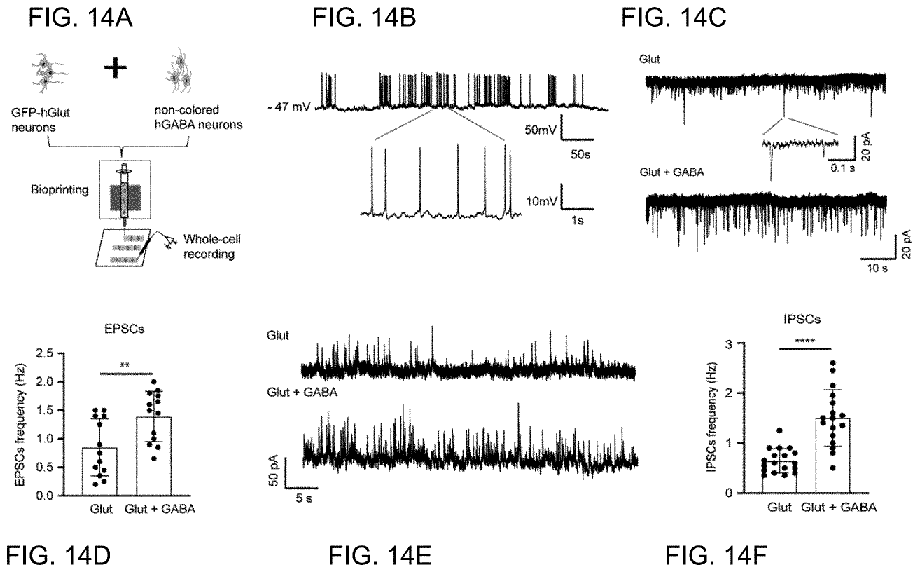

The printed neural tissues permit functional assessment such as by electrophysiological recording. GFP-glutamatergic cortical progenitors were printed with non-colored MGE GABAergic progenitors as shown in FIG. 14. Whole-cell patch clamping indicated that the glutamatergic neurons exhibited spontaneous action potentials (sAP) about 3 weeks after printing as shown in FIG. 14B. Importantly, the glutamatergic neurons displayed excitatory and inhibitory postsynaptic currents (EPSCs & IPSCs), suggesting formation of functional networks between glutamate neurons and GABA interneurons. Moreover, these cells received more EPSCs and IPSCs when printing together with GABAergic neurons. These results confirmed the functional maturation of cortical projection neurons and GABAergic interneurons and the formation of functional neural networks in the printed tissues.

Whole-cell patch-clamp recordings were made from human PSC-derived cortical glutamatergic and GABAergic neurons. The bath solution consisted of 135 mM NaCl, 3 mM KCl, 2 mM CaCl2, 1 mM MgCl2, 10 mM HEPES, 11 mM glucose, 10 mM sucrose, pH 7.4. Recording pipettes were filled with an intracellular solution containing 120 mM potassium D-gluconate, 1 mM ethylene glycol-bis (β-aminoethyl ether) N,N,N′,N′-tetraacetic acid (EGTA), 10 mM 4-(2-hydroxyethyl)piperazine-1-ethanesulfonic acid (HEPES), 4 mM ATP-Mg, 0.3 mM GTP-Na, 10 mM phosphocreatine, 0.1 mM CaCl2, 1 mM MgCl2, pH 7.2, 280-290 mOsm/L. Briefly, the neurons were held at −70 mV to record the Na+/K+ channel activities with voltage-clamp model. For recording action potentials, the cells were held at 0 pA with the current-clamp model, and with the steps of injected currents from −50 pA to +50 pA. Spontaneous excitatory postsynaptic currents (sEPSCs) and spontaneous inhibitory currents (sIPSCs) were recorded in gap-free mode at a holding potential of −70 mV and 0 mV, respectively. sEPSCs were blocked by application of the glutamate receptor antagonist 6-Cyano-7-nitroquinoxaline-2,3-dione (CNQX, 10 μM) and D-(−)-2-amino-5-phosphonopentanoic acid (D-APV, 50 sIPSCs were blocked by application of the GABAA receptor antagonist bicuculline (10 An Olympus BX51WI microscope was used to visualize neurons. A MultiClamp 700B amplifier (Axon instruments, Molecular Devices, Sunnyvale, Calif., USA) was used to investigate the voltage clamp and current clamp recordings. Signals were filtered at 4 kHZ using a Digidata 1550B analog-digital converter (Axon instruments) and stored for further analysis. Data were analyzed with Clampfit 11.0.3 (Axon instruments), GraphPad Prism 5 (GraphPad Software Inc., La Jolla, Calif., USA), CorelDraw 2019 (Corel, Canada), Igor 4.0 (WaveMetrics, Lake Oswego, Oreg., USA).

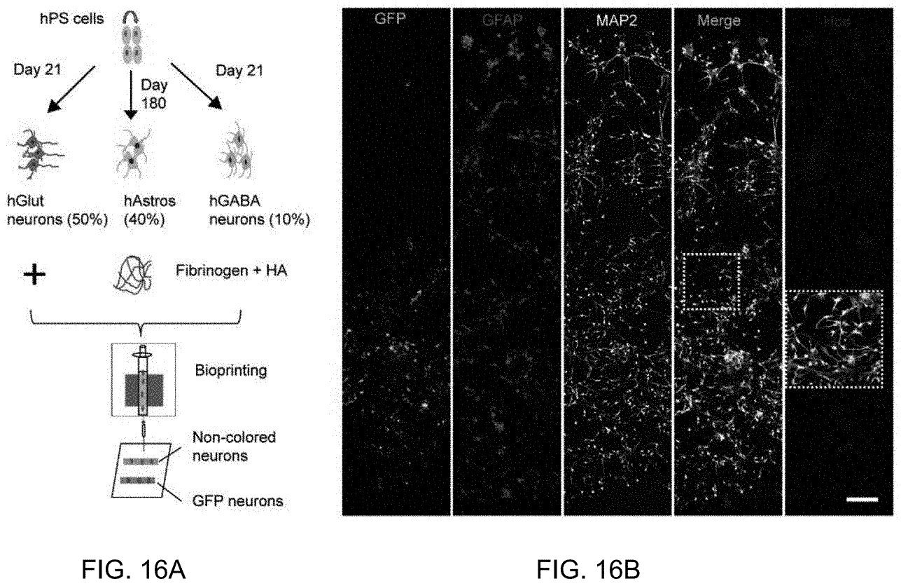

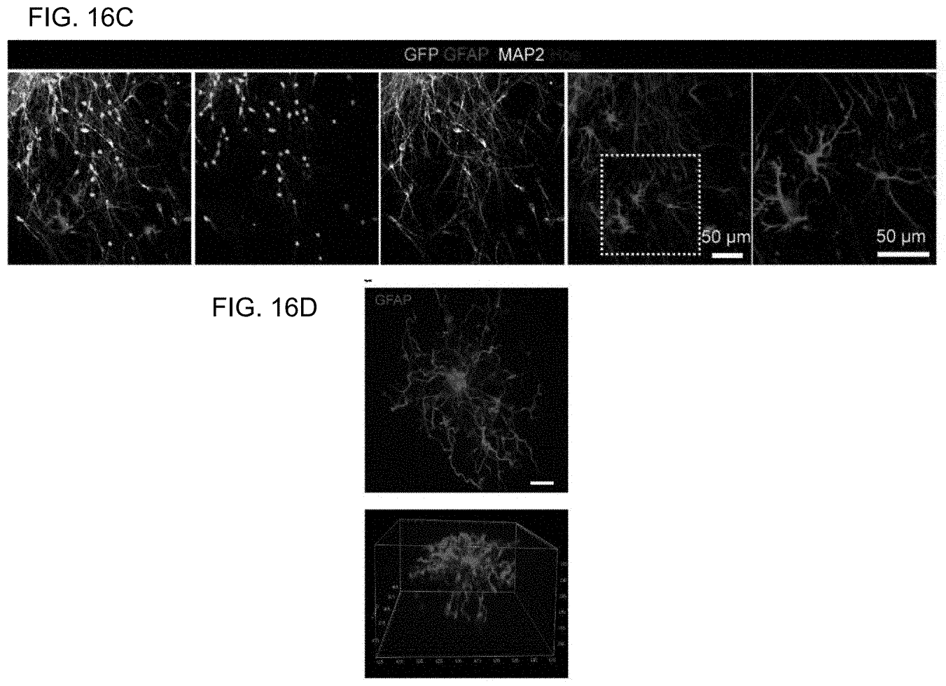

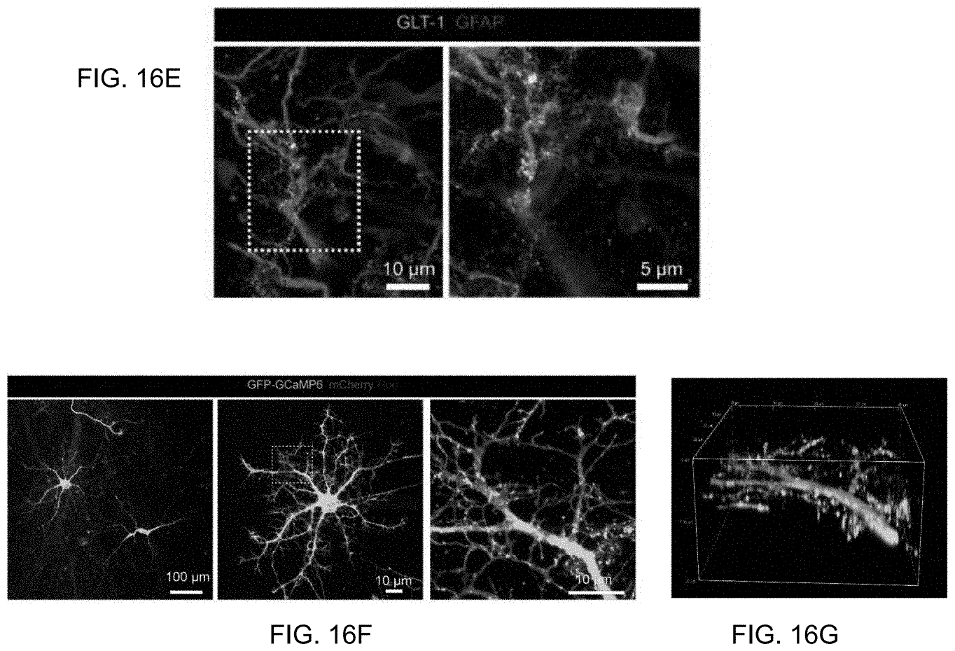

Appropriate neuronal network function requires the presence of glia, including astrocytes. Next, hPSC-derived astrocyte progenitors were incorporated into the above glutamate neurons and GABA interneurons at a ratio of 4:5:1. A two-layered tissue was also printed consisting of GFP⁺ and GFP⁻ cells; and each layer had the three cell types as shown in FIG. 16. The GFAP⁺ astrocytes and the MAP2⁺ neurons were distributed throughout the printed tissue.

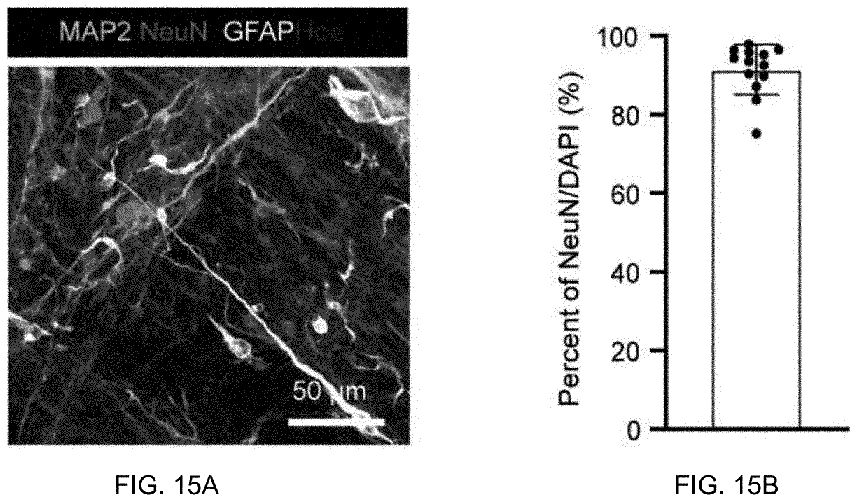

In the prints with both neurons and astrocytes, most of the neurons became NeuN⁺ neurons (>90%) at day 30 post-printing as shown in FIG. 15, indicating the enhanced neuronal maturation in the printed tissue. Particularly, astrocytes became progressively mature with more elaborate processes, which is not often seen in 2D cultures. While GFAP staining showed the extensive branches of astrocyte processes, GFP labeling revealed fine processes and their sheets. Notably, 3D visualization showed that the astrocytic branches were intertwined with the mCherry⁺ neuronal processes, suggesting close neuron-astrocyte interactions.

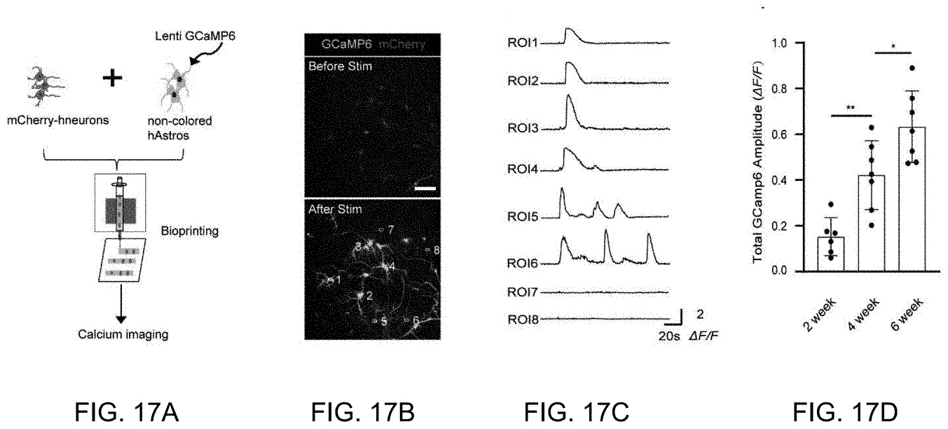

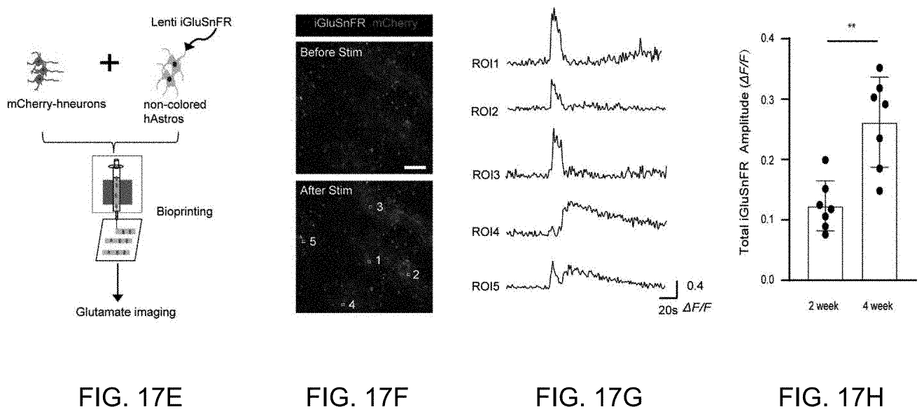

Astrocytes, in response to neuronal signals, generate calcium signals, which enable them to modulate neuronal network. To determine if the astrocytes functionally integrate into the printed neural networks, mCherry-neurons were printed with astrocytes that were infected with GCaMP6, a fluorescent calcium sensor as shown in FIG. 17. The application of high concentration KCl solution (67 nM), which depolarizes neurons, elicited calcium responses in astrocytes in the printed tissues. The total ΔF/F of GCaMP6 signals increased from 2 to 6 weeks after printing. Thus, the neurons and astrocytes in the printed tissues were functionally connected.

One of the most important functions of astrocytes is to recycle neurotransmitters like glutamate that are released to the synaptic cleft. Indeed, the astrocytes, printed from non-colored hPSCs expressed GLT-1 (glutamate transporter 1) or excitatory amino acid transporter 2 (EAAT2) at day 40 post-printing as shown in FIG. 16E, suggesting maturation of astrocytes. Networks between neurons and astrocytes were examined using live imaging of glutamate indicator iGluSnFR as shown in FIG. 17. Again, when applying KCl solution, which triggers cortical neurons to release glutamate, the iGluSnFR signals in astrocytes were induced. The total ΔF/F of iGluSnFR signals increased from 2 to 4 weeks after printing. These observations indicated that astrocytes uptake glutamate released from neurons in the printed tissues.

The calcium and glutamate live imaging were conducted as following. Briefly, Human PSC-derived astrocyte progenitors at day 180 were infected with Lenti-GCaMP6 or Lenti-iGluSnFR. After 3 days, the virus infected astrocytes were then printed with hPSC-derived cortical and MGE progenitors at ratio of 4:5:1. Calcium or glutamate imaging was performed as described (Sloan, S. A. et al. Neuron 95, 779-790 e776, doi:10.1016/j.neuron.2017.07.035 (2017)). Briefly, the printed tissues were washed with low potassium Tyrode's solution (Low-KCl) (2 mM KCl, 129 mM NaCl, 2 mM CaCl₂, 1 mM MgCl₂, 30 mM glucose, 25 mM HEPES, 0.1% and Bovine Serum Albumin, pH 7.4) three times and incubated with the solution for 30 min at 37° C. When imaging, the sample was placed on the stage of confocal fluorescence microscope (A1, Nikon). A high potassium Tyrode's solution (High-KCl) (67 mM KCl, 67 mM NaCl, 2 mM CaCl₂, 1 mM MgCl₂, 30 mM glucose, 25 mM HEPES, 0.1% and Bovine Serum Albumin, pH 7.4) was then applied. As a control, the virus infected astrocytes progenitors were also cultured alone and High-KCl solution was applied. ImageJ was used for the following analysis. The fluorescence change was defined as ΔF/F(t)=(F₀−F(t))/F₀, where F₀ is the average fluorescence intensity of the imaging area for samples in the Low-KCl solution, F(t) was the fluorescence intensity at given time. The ΔF/F of printed tissue was normalized by comparison of the ΔF/F of control.

### Example 2: Neuron-Astrocyte Interaction in the Prints in Alexander Disease

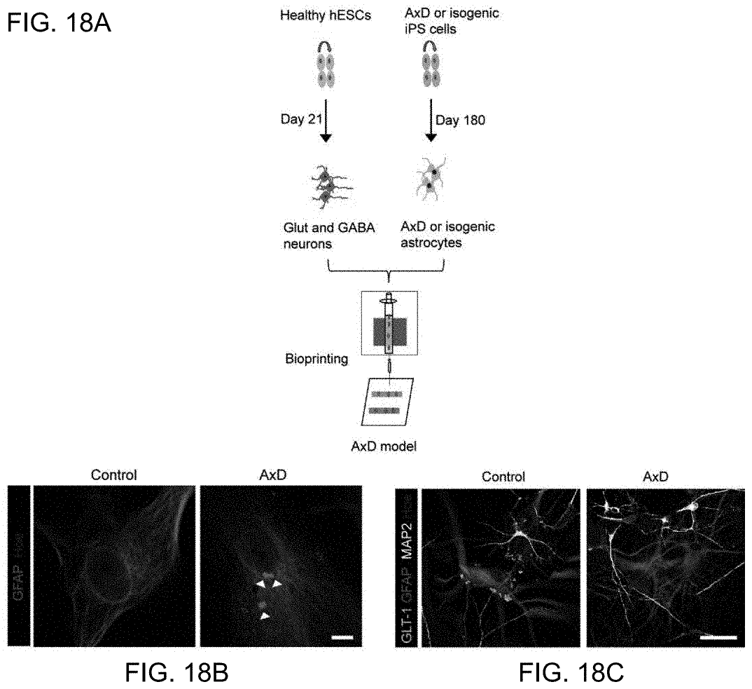

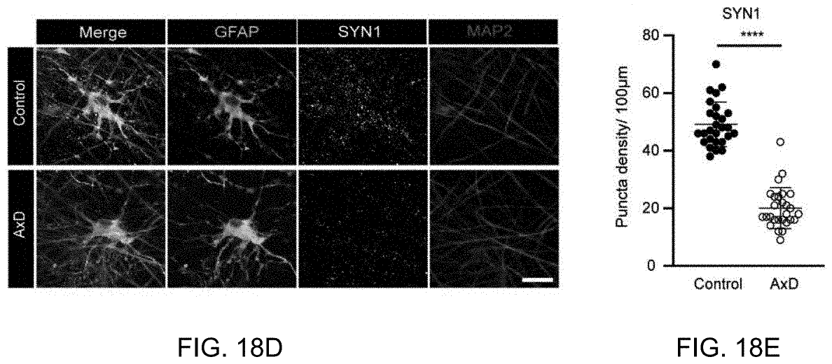

The functional neuron-glial networks in the prints present an ideal model for examining pathological processes. Taking Alexander disease (AxD) as an example, a neurodegenerative disease caused by mutations in the glial fibrillary acidic protein (GFAP) gene, neuron-astrocyte interaction was assessed in the prints similar to what is described in FIG. 16. In this case, the (glutamate and GABA) neurons were derived from normal hESCs (H9) whereas the astrocytes were from either AxD (R88C mutation) or isogenic (mutation corrected by CRISPR) iPSCs (Jones, J. R. et al. Cell Rep 25, 947-958 e944, doi:10.1016/j.celrep.2018.09.083 (2018)). After printing, compared to the samples from isogenic control the AxD astrocytes displayed GFAP aggregation intracellularly as shown in FIG. 18, which recapitulates the pathological feature of AxD. The printed AxD tissues showed less expression of GLT-1 comparing to control at day 20 post-printing. By 30 days after printing, MAP2⁺ neurons and GFAP⁺ astrocytes displayed a complex morphology with elaborate processes and expression of synapsin. Interestingly, the AxD tissues had a significantly reduced synaptic puncta density than the tissue with isogenic astrocytes, suggesting the less supportive effect of AxD astrocytes on synaptogenesis.

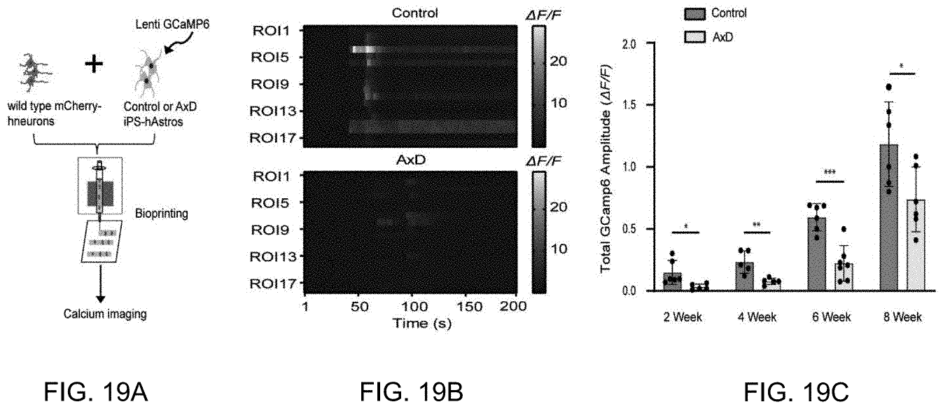

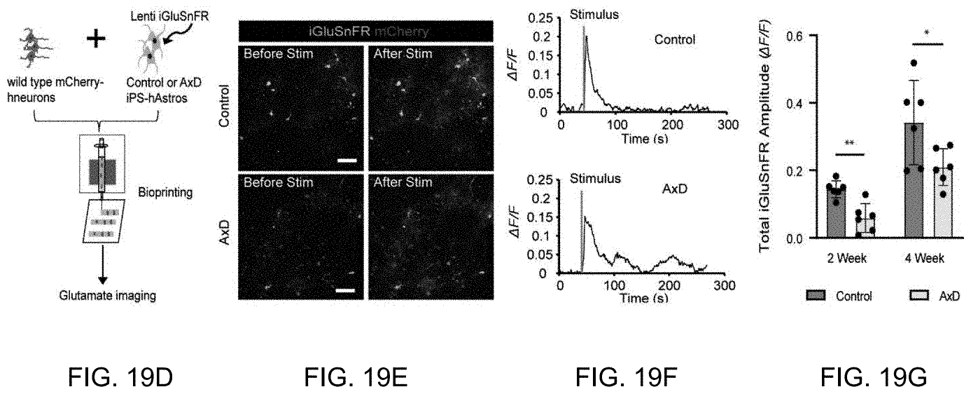

It was previously shown, AxD patient astrocytes failed to propagate calcium waves in pure 2D cultures (Jones et al. Cell Rep 25, 947-958 e944, doi:10.1016/j.celrep.2018.09.083 (2018)). To determine if AxD astrocytes alter their response to neuronal function, prints were used with AxD or isogenic astrocytes which were previously infected with GCaMP6 as shown FIG. 19. High KCl (67 nM), which stimulates neuronal function by inducing depolarization, elicited a robust Ca⁺ response in isogenic astrocytes. In contrast, AxD astrocytes showed significantly fewer calcium responses than the isogenic control. The total ΔF/F of GCaMP6 signals for AxD astrocytes were much lower following KCl stimulation as compared to that of control overtime. The neuronal-glial connections in AxD were investigated using live imaging of glutamate by iGluSnFR as shown in FIG. 19. Upon neuronal stimulation with high KCl, the AxD tissues exhibited fewer changes of iGluSnFR signals compared to the isogenic control. The total ΔF/F of iGluSnFR signals for AxD astrocytes were significantly lower from 2 to 4 weeks after printing as compared to those of the isogenic tissues. These results indicate that the disease relevant functional phenotypes are readily displayed in the printed human neural tissues.

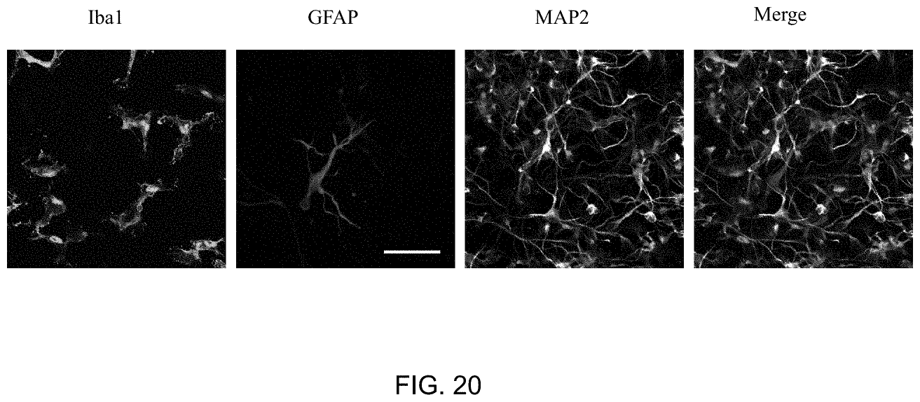

Microglia were also printed into the neural tissues as shown in FIG. 20. The immunostaining showed microglia, neurons and astrocytes were survived in the tissues.

### Example 3: Differentiating Functional Brain Regional-Specific Tissues

The reagents and methods comprising the system disclosed herein were used to fabricate human cortical-striatal tissue to mimic the cortical-striatal circuits in human being brain by printing cortical projections neurons with DARPP32⁺ striatal medium spiny neurons.

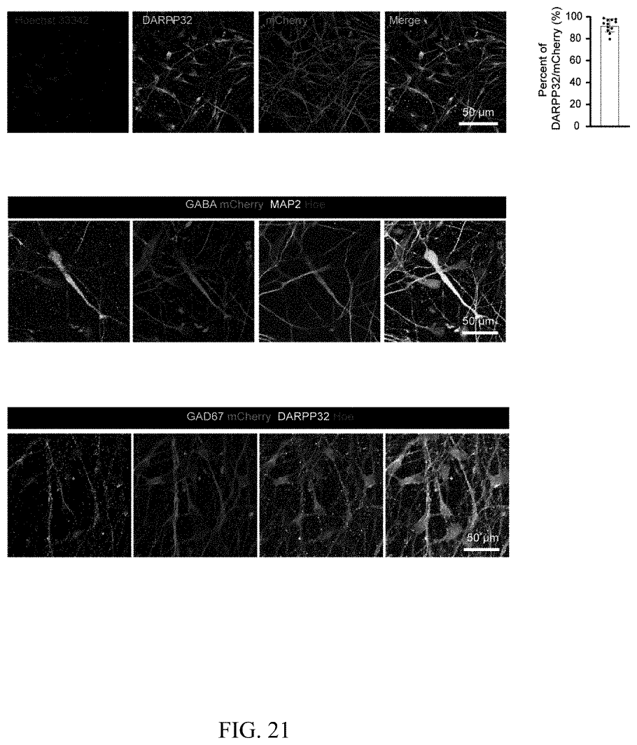

Striatal DARPP32⁺ neurons or medium spiny neurons were generated as set forth in Ma et al., 2012 (Cell Stem Cell 10: 455.464). Briefly, 40 ng/mL recombinant human sonic hedgehog (Shh C25II) N terminus protein (R&D Systems, Cat. #464-SH-025/CF) was added to neural differentiation medium from day 7 to day 25 after one week of dual SMAD (small mothers against decapentaplegic family) signaling inhibitors treatment. At day 25, these striatal progenitors were treated with valproic acid (VPA) (10 μM, Sigma) for 5 days and single striatal neurons were prepared for printing. These stem cells-derived striatal neurons showed expression of GABA, CAD67 with a high level of DARPP32 (>90%) as shown after immunofluorescence microphotography in FIG. 21A-FIG. 21C.

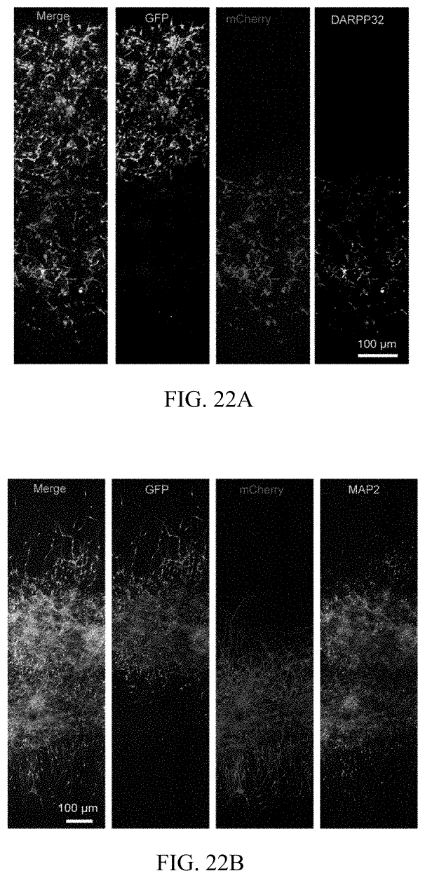

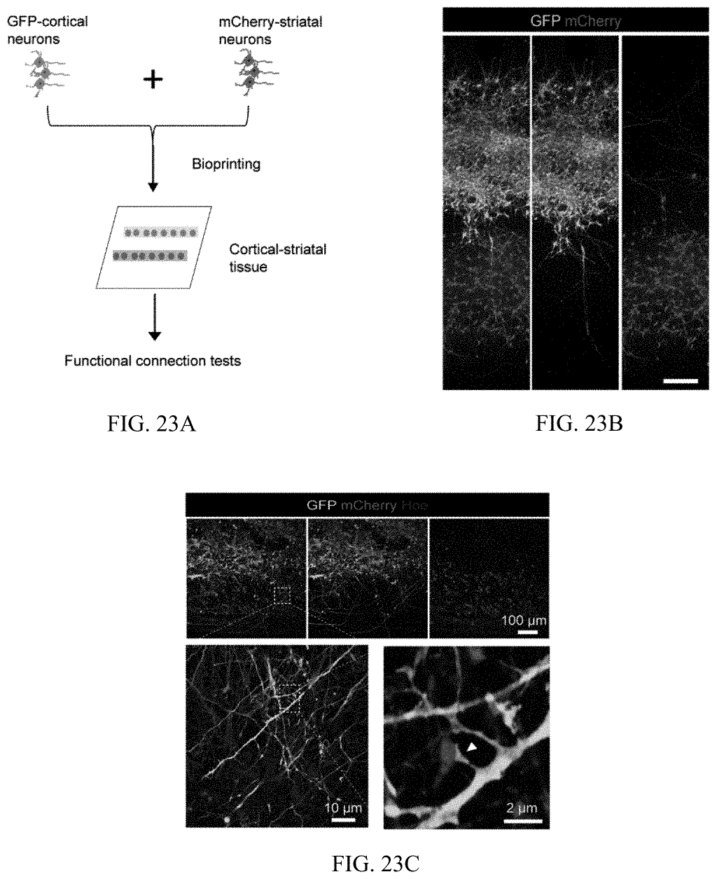

Neurons not only synapse each other in the same brain region but also connect to each other between nuclei or layers. After printing, cortical neurons expressed GFP, and striatal neurons, expressing mCherry and DARPP32, showed a distinguished tissue separation. Both neuronal types became mature with expression of MAP2 at 2 weeks of printing as shown in FIG. 22B, which illustrates the experimental protocol for bioprinting GFP-cortical neurons and mCherry-striatal neurons. The printed cortical and striatal neuronal layers were well-maintained 15 days after printing, and cortical neurons projected toward striatal neuronal layer while the striatal neurons extended their processes toward the cortical neuronal domain as shown in FIG. 23B. The projected cortical neurites (axons) formed physical contacts with the striatal neurites, indicating the formation of connection.

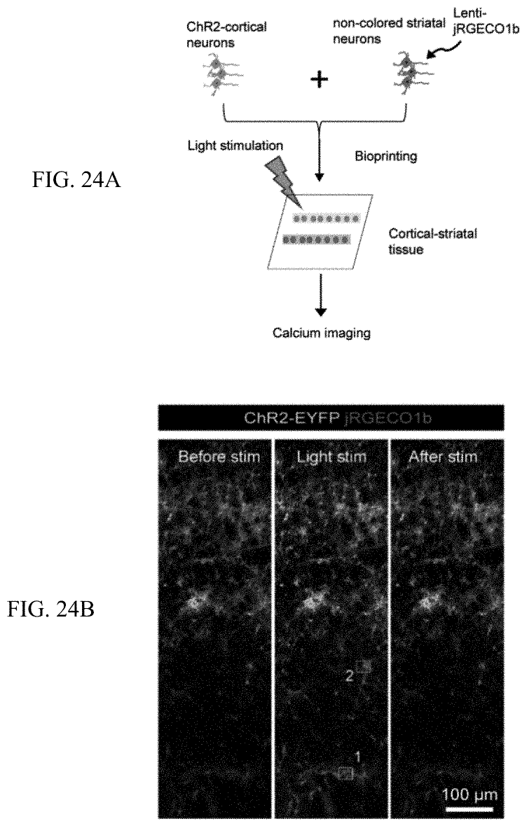

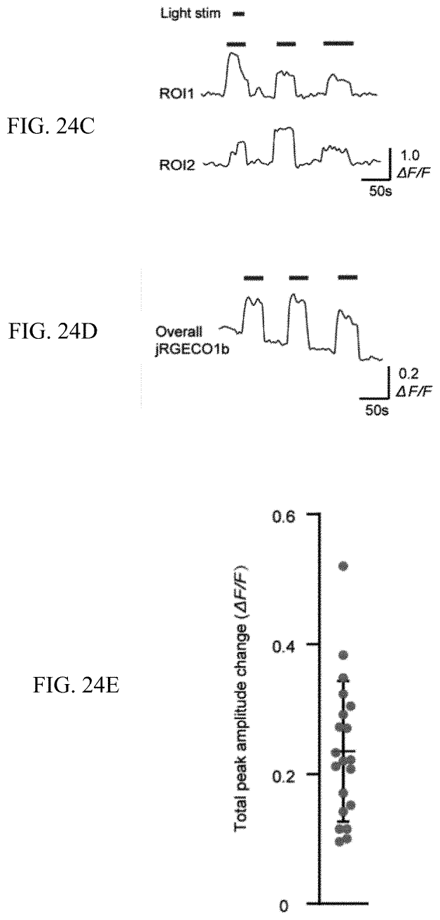

To determine whether neurons in the printed cortical tissue formed functional synaptic connections with those in the striatal tissue, cortical neurons were printed that expressed ChR2-EYFP (see, Dong et al., 2020, iScience 23: 100829), with striatal neurons that expressed red calcium indicator jRGECO1b (Dana et al., 2016, Elife 5). Application of 470-nm light was found to stimulate ChR2-EYFP cortical neurons elicited calcium responses in the striatal neuron layer 2 weeks after printing as showing in FIG. 24. Even striatal neurons far from the cortical layer showed increased calcium responses. The total ΔF/F of calcium signals for the whole striatal neuron layer significantly increased with stimulation as showing in FIG. 24E, suggesting functional connections of the cortical neuron layer with the striatal neuron layer were produced in these printed tissues.

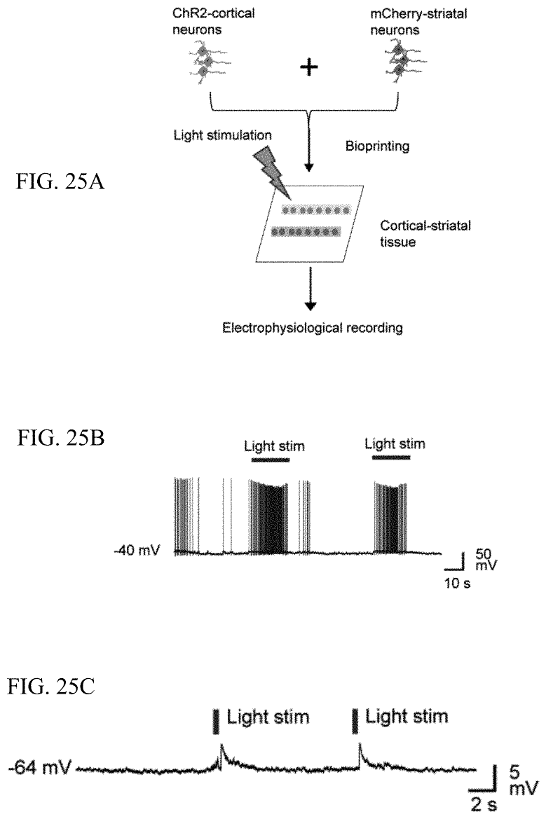

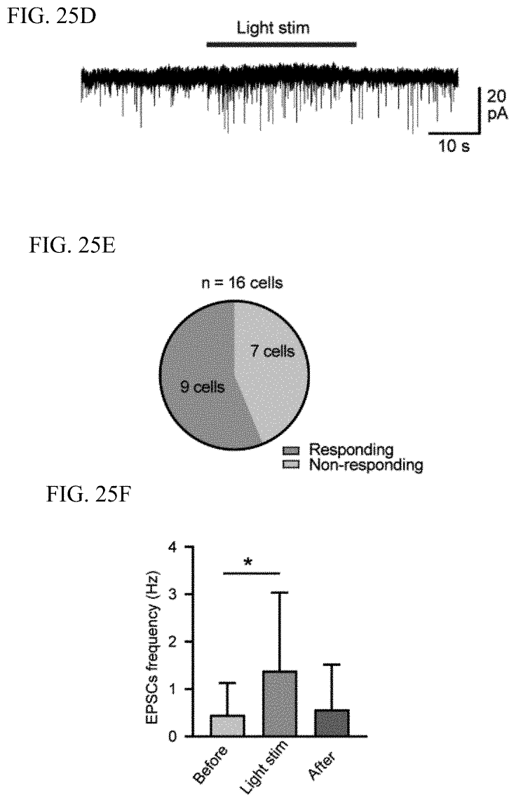

The functional connections of printed cortical-striatal tissue were further characterized by electrophysiological recording. As showing in FIG. 25A, cortical neurons were printed that expressed ChR2-EYFP, with striatal neurons that expressed mCherry. For ChR2 stimulation, a light stimulation fiber was placed 5 mm from the dish. A custom-made LED device (1 Watt, 470 nm; Cree lighting Inc.) coupled to a fiber optic cable was used to achieve the stimulation. For printed cortical-striatal tissue about 5 weeks after printing, spontaneous action potentials, EPSPS and EPSCs were recorded before the blue light stimulation(baseline), with blue light stimulation (30˜60 s, 470 nm, 0.4 mW/mm²) and after blue light stimulation under the voltage-clamp mode. mCherry-striatal neurons in printed cortico-striatal tissue were randomly selected for recordings. More than 56% striatal neurons exhibited responses with light stimulation as showing in FIG. 25E. And the frequency of EPSCs increased with stimulation, indicating the functional network formation in printed cortical-striatal tissue.

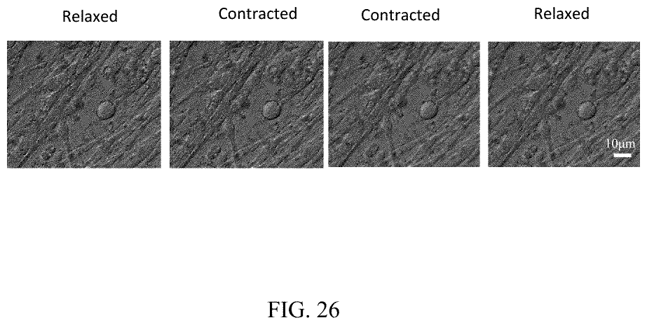

FIG. 26 show muscle cells with printed cortical-muscle tissue contracted and relaxed after one month of printing, as indicated by the red arrows in FIG. 28. Thus, printed cortical-muscle tissue according to this invention exhibited biological functions, indicating that the invention can be utilized to assemble multiple brain-regional specific tissues with other tissues to form functional connectivity.

All publications, patents, and patent applications mentioned in this specification are herein incorporated by reference to the same extent as if each individual publication, patent, and patent application was specifically and individually indicated to be incorporated by reference. Those skilled in the art will recognize or be able to ascertain using no more than routine experimentation many equivalents to the specific embodiments described herein. The scope of the present embodiments described herein is not intended to be limited to the above description, but rather is as set forth in the appended claims. Those of ordinary skill in the art will appreciate that various changes and modifications to this description can be made without departing from the spirit or scope of the present invention, as defined in the following claims. Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description can be made without departing from the spirit or scope of the present invention, as defined in the following claims.

We claim:

1. A method of generating a neural tissue construct, comprising:
(a) horizontally depositing by bioprinting on a suitable surface a band comprising a mixture, wherein the mixture comprises
(i) glutamatergic cortical progenitor cells;
(ii) GABAergic interneuron progenitor cells; and
(iii) bio-ink
(b) repeating the depositing step of (a) a plurality of times to form a plurality of bands; and
(c) maturing the deposited plurality of bands to allow the cells to form a neural tissue construct.

2. The method of claim 1, wherein the bio-ink comprises a hydrogel and hyaluronic acid.

3. The method of claim 2, wherein the hydrogel comprises fibrinogen and thrombin.

4. The method of claim 3, wherein the hydrogel further comprises a protease inhibitor.

5. The method of claim 4, wherein the hydrogel is crosslinked using a crosslinking agent after each of the depositing steps.

6. The method of claim 5, wherein the crosslinking agent comprises buffer CaCl₂, thrombin and transglutaminase (TG).

7. The method of claim 1, wherein the plurality of bands are physically separated.

8. The method of claim 1, wherein each of the plurality of bands have a thickness horizontally of about 50 μm.

9. The method of claim 1, wherein the glutamatergic cortical progenitor cells and the GABAergic interneuron progenitor cells are present in the mixture at a ratio of 4:1.

10. The method of claim 1, wherein the glutamatergic cortical neural progenitor cells are 21-day human pluripotent stem cells (hPSCs)-derived glutamatergic cortical neural progenitor cells.

11. The method of claim 1, wherein the GABAergic interneuron progenitor cells are 21-day human pluripotent stem cells (hPSCs)-derived GABAergic interneuron progenitor cells.

12. The method of claim 1, wherein the mixture further comprises astrocyte progenitor cells.

13. The method of claim 12, wherein the glutamatergic cortical progenitor cells, the GABAergic interneuron progenitor cells and the astrocyte progenitor cells are present in the mixture at a ratio of 5:1:4.

14. The method of claim 12, wherein the mixture further comprises microglial cells.

15. The method of claim 1, wherein the neural tissue construct comprises a functional neural network.

16. The method of claim 15, wherein the functional neural network is formed within about 2 weeks to about 4 weeks.

17. The method of claim 13, wherein the neural tissue construct comprises a functional neural-glia network.

18. The method of claim 17, wherein the functional neural-glia network is formed within about 2 weeks to about 4 weeks.

19. The method of claim 1, wherein the maturing occurs in neuronal basal medium comprising at least one of 2% B-27, a Brain Derived Neurotrophic Factor (BDNF), a Glial cell line-derived neurotrophic factor (GDNF), ROCK inhibitor, or γ-secretase and notch pathway inhibitor.

20. A neural tissue construct produced by the method of claim 1.

21. A method of screening a test agent, the method comprising:
(a) depositing a test agent on the neural tissue construct of claim 1;
(b) measuring a functional parameter of the contacted neural tissue construct; and
(c) comparing the functional parameter to that parameter measured in neural tissue construct which has not been contacted with the test agent, wherein modulation of the functional parameter after contact with the test agent indicates the test agent is a candidate therapeutic agent.

22. The method of claim 21, wherein the test agent is (i) an organic compound; (ii) a nucleic acid; (iii) a peptide; (iii) a polypeptide; or (iv) an antibody.

23. The method of claim 1, wherein the neural tissue construct further comprises DARPP32⁺ striatal medium spiny neurons.

24. The method of claim 23, wherein cortical neurons in the neural tissue construct express ChR2-EYFP and striatal neurons express red calcium indicator jRGECO1b.

25. A neural tissue construct produced by the method of claim 23.

26. The method of claim 21, where the cells of the neural tissue construct express a detectable marker that is responsive to the test agent.

27. The method of claim 26, wherein the detectable markers include but are not limited to green fluorescent protein (GFP), mCherry, and ChR2-EYFP, or red calcium indicator jRGECO1b.
